(19) United States
(12) Patent Application Publication
(10) Pub. No.: US 2021/0010064 A1
(43) Pub. Date: Jan. 14, 2021

(54) ENRICHMENT OF NUCLEIC ACIDS

(71) Applicants: THE TRUSTEES OF THE UNIVERSITY OF PENNSYLVANIA, Philadelphia, PA (US); WAGENINGEN UNIVERSITEIT, Wageningen (NL)

(72) Inventors: Haim H. BAU, Swarthmore, PA (US); Jinzhao SONG, Philadelphia, PA (US); Changchun LIU, Bala Cynwyd, PA (US); Michael G. MAUK, Philadelphia, PA (US); John VAN DER OOST, Renkum (NL); Jorrit WIETZE HEGGE, Wageningen (NL)

(21) Appl. No.: 16/980,083

(22) PCT Filed: Mar. 14, 2019
(86) PCT No.: PCT/US19/22255

§ 371 Date: Sep. 11, 2020

Related U.S. Application Data
(60) Provisional application No. 62/642,984, filed on Mar. 14, 2018.

(51) Int. Cl. C12Q 1/6806; C12N 15/66; C12N 15/10; G01N 33/569; C12N 9/22

(52) U.S. Cl. CPC C12Q 1/6806; C12N 15/66; C12N 15/1003; C12Q 1/686; C12N 9/22; C12N 2310/20; C12Q 2531/113; G01N 33/56983

(57) ABSTRACT

Provided are methods directed to enriching nucleic acids in a biological sample. These methods, in some embodiments can discriminately enrich the abundance of low-copy nucleic acids relative to higher-copy nucleic acids. In some embodiments, the methods provided can enrich a low-copy number mutant allele associated with a disease state, thus allowing early detection and optimized treatment. In other embodiments, the methods can be used for detection of particular molecules, such as antigens, in a sample.

### CROSS REFERENCE TO RELATED APPLICATIONS

This application claims priority to and the benefit of U.S. Provisional Patent Application No. 62/642,984, filed Mar. 14, 2018, the disclosure of which is incorporated herein by reference in its entirety.

### SEQUENCE LISTING

This application contains a Sequence Listing which has been submitted electronically in ASCII format and is hereby incorporated by reference in its entirety. Said ASCII copy, created on Mar. 12, 2019, is named 104377_000221 and is 40,814 bytes in size.

### FIELD OF THE INVENTION

Disclosed herein are methods of enriching nucleic acids.

### BACKGROUND OF THE INVENTION

Amplification of low-copy number nucleic acids in a sample comprising similar sequences to high-copy number nucleic acids remains a significant technical challenge. Because high-copy number nucleic acids can outcompete and sequester reagents necessary for amplification, low-copy numbers are often undetected, which can result in delayed diagnoses, incomplete data for genetic studies, or failure to identify clinically relevant biomarkers.

Recent progress in tumor genotyping facilitates identification of oncogenic mutations responsible for the initiation and maintenance of cancer and mechanisms of resistance to targeted therapeutics. The ever-expanding pharmacopeia of oncologic therapies that target specific cancer mutations require sensitive, non-invasive methods for cancer allele detection to select effective therapy (Oxnard, Geoffrey R., et al. “Noninvasive detection of response and resistance in EGFR-mutant lung cancer using quantitative next-generation genotyping of cell-free plasma DNA.” Clinical cancer research 20.6 (2014): 1698-1705.). Such therapeutics improve outcomes and reduce adverse effects and cost, especially when effective treatment options are identified early in the progression of an aggressive cancer as patient survival rates can diminish quickly over time. Biomarkers obtained from a patient can be used to better understand a tumor's genetics, susceptibility to drugs, and drug-resistance, as well as an early diagnosis.

Tumor nucleic acids can harbor biomarkers that are informative of the nature of the tumor and the cells residing therein. To obtain these tumor nucleic acids, invasive tissue biopsies that can require surgery are often required, but some patients are not even candidates for such biopsies due to poor health and/or inaccessible tumor location. Moreover, tumor biopsy provides only localized samples that do not represent the full spectrum of cancer-related mutations. An alternative to tissue biopsy is liquid biopsy (LB), a minimally invasive and relatively inexpensive technique of testing blood or urine from a subject for cell-free circulating tumor DNA or RNA (cf-ctDNA or cf-ctRNA, respectively). LB provides a source of fresh tumor-derived material and downstream assays that detect biomarkers provide valuable information pertaining to cancer genotypes.

Sensitive genotyping assays, such as targeted next-generation sequencing (NGS), PCR that suppresses wild type DNA amplification with peptide nucleic acid (PNA)-clamping, digital drop PCR (ddPCR) with and without multiplexed preamplification, and Cancer Personalized Profiling by deep Sequencing (CAPP-Seq) are used to identify mutant alleles. But because biomarkers for undiagnosed cancers can be rare mutants, their detection is often masked by the wildtype allele, which makes it necessary to augment these assays by removing the wildtype allele that results in a relative enrichment of the mutant alleles prior to (or during) amplification and/or sequencing.

Thus, there is a need for methods of enriching nucleic acids in a biological sample. The disclosed methods are directed to these and other important needs.

### SUMMARY OF THE INVENTION

Disclosed herein are methods of enriching a target nucleic acid in a sample comprising contacting the sample with a guide nucleic acid having a sufficiently complementary sequence to a non-target nucleic acid to allow hybridization of the guide nucleic acid and the non-target nucleic acid to form a guide/non-target hybrid; contacting the sample with an endonuclease having an affinity for the guide/non-target hybrid; and amplifying the target nucleic acid.

Methods are provided for enriching a target nucleic acid in a sample comprising contacting the sample with a guide nucleic acid having a sufficiently complementary sequence to a non-target nucleic acid to allow hybridization of the guide nucleic acid and the non-target nucleic acid to form a guide/non-target hybrid; contacting the sample with an endonuclease having an affinity for the guide/non-target hybrid; and incubating the sample.

Also disclosed herein are methods of detecting the presence or absence of cell-free circulating tumor nucleic acids (cf-ctNA) in a sample from a subject, comprising contacting the sample with a guide nucleic acid having a sufficiently complementary sequence to a non-cf-ctNA to allow hybridization of the guide nucleic acid and the non-cf-ctNA to form a guide/non-cf-ctNA hybrid; contacting the sample with an endonuclease having an affinity for the guide/non-cf-ctNA hybrid under conditions suitable for the endonuclease to cleave the non-cf-ctNA; amplifying the ct-cfNA, if any, in the sample; and detecting the presence or absence of cf-ctNA.

Methods are also provided for detecting a molecule in a sample, comprising contacting the sample with a first antibody having an affinity for a first epitope on the molecule, wherein in the presence of the molecule a molecule-first antibody complex is formed; contacting the sample with a probe comprising (i) a second antibody having an affinity for a second epitope on the molecule, (ii) a guide nucleic acid, and (iii) optionally a linker linking the second antibody to the guide nucleic acid, wherein in the presence of the target molecule-first antibody complex form a complex of the probe and the target molecule-first antibody complex is formed; contacting the sample with a target nucleic acid comprising a first portion labeled with a dye, a second portion labeled with a quencher, and a sequence at least partially complementary to a sequence of the guide nucleic acid, wherein in the presence of the detectable complex, the guide nucleic acid of the probe hybridizes to the target nucleic acid to form a guide-target complex; contacting the sample with an endonuclease having an affinity for the guide-target complex; and detecting a signal related to the dye.

### BRIEF DESCRIPTION OF THE DRAWINGS

The patent or application file contains at least one drawing executed in color. Copies of this patent or patent application publication with color drawings will be provided by the Office upon request and payment of the necessary fee.

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIGS. 1A-C illustrate one example of the methods of the present disclosure directed to enriching nucleic acids. FIG. 1A depicts the sequences surrounding the G12 codon of wildtype and G12D mutant KRAS (FIG. 1A discloses SEQ ID NOS 1-2, respectively, in order of appearance); FIG. 1B illustrates the probe design and its interaction with the wildtype DNA (FIG. 1B discloses SEQ ID NOS 3-6, respectively, in order of appearance), and FIG. 1C illustrates the probe interaction with KRAS G12D mutant DNA (FIG. 1C discloses SEQ ID NOS 7-10, respectively, in order of appearance).

FIG. 2A is an image of gel electrophoresis of WT KRAS and mutant (KRAS G12D) incubated with Thermus thermophilus Argonaute (TtAgo) for 20 and 40 min. The WT KRAS is cleaved into two segments (54nt and 46nt) while the mutant (110nt) is spared. FIG. 2B is an electropherogram depicting Sanger sequencing without (left) and with (right) 20 min enrichment of DNA isolated from a cancer patient's blood. The invisible KRAS G12 (<1%) in the unenriched sample is readily detectable after TtAgo enrichment. FIG. 2B discloses SEQ ID NOS 11-12, respectively, in order of appearance.

FIG. 3A depicts ddPCR results of a heterogeneous sample comprising wildtype KRAS G12 and mutant KRAS G12D, wherein the sample was not subjected to a PfAgo-mediated cleavage assay. FIG. 3B depicts flow cytometry data of a heterogeneous sample comprising wildtype KRAS G12 and mutant KRAS G12D, wherein the sample was subjected to a PfAgo-mediated cleavage assay.

FIG. 4A illustrates the design of guide nucleic acids for epidermal growth factor receptor (EGFR). FIG. 4A discloses SEQ ID NOS 13-21, respectively, in order of appearance. FIG. 4B shows images of gel analyses of PfAgo-mediated cleavage assays of mutant and wildtype EGFR using different guide nucleic acids and different reaction conditions.

FIG. 5A depicts the guide nucleic acid design for different strains of the zika virus. FIG. 5A discloses SEQ ID NOS 22-25, respectively, in order of appearance. FIG. 5B is an image of gel electrophoresis analysis of a cleaving assay of different strains of the zika virus.

FIG. 6 graphically depicts an antigen detection assay.

FIG. 7A-FIG. 7D show cleavage efficiency of WT KRAS and KRAS G12D DNA and RNA in Buffers 1, 2, 3, and S (Table 1) at 80° C. FIG. 7A: DNA cleavage as a function of buffer composition. FIG. 7B: DNA cleavage in Buffer 2 as a function of added [Mg2+] in the absence and presence of betaine. FIG. 7C: RNA cleavage as a function of buffer composition. FIG. 7D: RNA cleavage in Buffer 2 as a function of added [Mg²⁺] in the absence and presence of betaine and dNTP. All experiments were carried out with KRAS Sense (S) strand and 16 nt KRAS-S guide with a pair mismatch at position 12 (MP12). Incubation time 20 min. TtAgo:guide:target=1:0.2:0.2. N=3.

FIG. 8A-FIG. 8B show the effects of dNTPs (FIG. 8A) and NTPs (FIG. 8B) on EGFR (L858R)-S RNA cleavage with guide EFGR (L858R)-S (16nt)-MP10 at 80° C. FIG. 8C shows the effect of pH on EGFR (L858R)-S DNA cleavage with guide EGFR (L858R)-S (16nt)-MP10 at 75° C. TtAgo:guide:target=1:0.2:0.2. N=3.

FIG. 9A-FIG. 9C show NAVIGATER's discrimination efficiency depends sensitively on guide-off target mismatch pair's position (MP). FIG. 9A: Overview of the KRAS-S guide and S target sequences. The various guides vary in the position of the pair mismatch between S gDNA and S KRAS G12D. FIG. 9B: Electropherograms (polyacrylamide urea gel) of NAVIGATER (80° C., 20 min) products of S WT KRAS DNA and S KRAS G12D DNA strands as functions of MP. FIG. 9C: Cleavage efficiencies of S WT KRAS DNA and S KRAS G12D DNA as a function of MP. All experiments were carried out with short guides (15/16 nt) in Buffer 3 at 80° C. TtAgo:guide:target=1:0.2:0.2. N=3.

FIG. 10 shows KRAS-antisense (AS) guide and AS target sequences. The various guides vary in the position of the pair mismatch between AS gDNA and AS KRAS G12D. FIG. 10 discloses SEQ ID NOS 26-38, respectively, in order of appearance.

FIG. 11 shows EGFR-guide and target sequences. The various guides vary in the position of the pair mismatch between gDNA and EGFR L858R. FIG. 11 discloses SEQ ID NOS 39-68, respectively, in order of appearance.

FIG. 12 shows EGFR-guide and target sequences. The various guides vary in the position of the pair mismatch between gDNA and EGFR T790M. FIG. 12 discloses SEQ ID NOS 69-92, respectively, in order of appearance.

FIG. 13 shows BRAF-guide and target sequences. The various guides vary in the position of the pair mismatch between gDNA and BRAF V600E. FIG. 13 discloses SEQ ID NOS 93-116, respectively, in order of appearance.

FIG. 14A-FIG. 14C show short DNA guides (15/16 nt) provide a better discrimination between WT and MA. FIG. 14A: Cleavage efficiency as a function of guide length at 70° C. and 75° C.: (i) WT KRAS and KRAS G12D, S-DNA and RNA and (ii) AS WT KRAS and KRAS G12D. FIG. 14B: Cleavage efficiency as a function of temperature with 18/19 nt long guide (i) WT KRAS and KRAS G12D S-DNA, (ii) WT KRAS and KRAS G12D RNA, and (iii) WT KRAS and KRAS G12D AS-DNA. FIG. 14C: Cleavage efficiency of WT KRAS and KRAS G12D (i) S-DNA and (ii) RNA as functions of temperature (16 nt long guide). Buffer 3. TtAgo:guide:target=1:0.2:0.2. N=3.

FIG. 15A-FIG. 15D show excess guide concentration provides high discrimination efficiency. dsDNA KRAS and KRAS G12D cleavage efficiencies as functions of temperature: FIG. 15A: TtAgo/(S-guide)/(AS-guide) concentration ratio: 1:1:1, 40 min and 2 h incubation time; FIG. 15B: TtAgo/(S-guide)/(AS-guide) concentration ratio: 1:0.2:0.2, 40 min and 2 h incubation time. Electropherograms of NAVIGATER's products as a function of incubation (83° C.) time: FIG. 15C: TtAgo/(S-guide)/(AS-guide) ratio 1:1:1 and FIG. 15D: TtAgo/(S-guide)/(AS-guide) ratio 1:10:10. All experiments were carried out in Buffer 3 with KRAS-S (16nt)-MP12 and KRAS-AS (15nt)-MP13 guides. N=3.

FIG. 16A-FIG. 16C show NAVIGATER enriches MAs harboring deletion mutations. FIG. 16A: Common EGFR exon19 deletion mutations. FIG. 16B: Target sequences and guides for enriching MAs containing EGFR exon19 deletion mutations. FIG. 16B discloses SEQ ID NOS 145-164, respectively, in order of appearance. FIG. 16C: Electropherograms of cleaving assay products of WT dsEGFR (80 bp) and dsEGFR exon19 deletion mutants. Incubation time 1 hour at 83° C. TtAgo/S-guide/AS-guide ratio 1:10:10. Synthetic dsDNAs harboring common EGFR exon19 deletion mutations were used.

FIG. 17A-FIG. 17B show CRISPR-Cas9 based dsDNA cleavage. FIG. 17A: Electropherograms of dsKRAS (100 bp) cleaving assay products. CRISPR/Cas9 nonspecifically cleaved dsMAs harboring KRAS G12D and G12V. FIG. 17B: Electropherograms of dsEGFR (100 bp) cleaving assay products. CRISPR/Cas9 nonspecifically cleaved dsMA harboring EGFR L858R, but specifically cleaved dsWT EGFR, while sparing dsMA harboring EGFR deletion mutation E746-A750 del(1). The crRNA sequences are listed in FIG. 17A and FIG. 17B. N=3.

FIG. 18 shows electropherograms of six pancreatic cancer patient's samples (Table 3) without enrichment (control), once enriched for 50 min and 2 h and twice-enriched.

FIG. 19A-FIG. 19G show NAVIGATER enhances sensitivity of downstream detection methods. FIGS. 19A and 19B show ddPCR of samples from pancreatic cancer patients containing KRAS mutants (Table 3): FIG. 19A: Fraction of droplets reporting mutant alleles. FIG. 19B: Increase in mutant allele fraction after NAVIGATER enrichment. FIG. 19C, FIG. 19D, FIG. 19E show PNA-PCR's amplification curves of pancreatic cancer patients' samples before (FIG. 19C) and after (FIG. 19D) NAVIGATER, and amplification threshold time as a function of mutant fraction (FIG. 19E). FIG. 19F shows PNA-LAMP of simulated RNA samples before and after NAVIGATER carried out with a minimally-instrumented, electricity-free Smart-Connected Cup (SCC)20 (inset). FIG. 19E shows Sanger sequencing before and after NAVIGATER when detecting simulated RNA samples. All the controls were pre-processed with NAVIGATER in the absence of TtAgo. N=3.

FIG. 20A-FIG. 20C show KRAS G12D guide screening and results using PfAgo. FIG. 20A shows sense and antisense guides. FIG. 20B and FIG. 20C show guide screening electropherograms results for sense guides and antisense guides, respectively.

FIG. 21A-FIG. 21C show BRAF V600E guide screening and results using PfAgo. FIG. 21A shows sense and antisense guides. FIG. 21B and FIG. 21C show guide screening electropherograms results for sense guides and antisense guides, respectively.

FIG. 22A-FIG. 22C show EGFR T790M guide screening and results using PfAgo. FIG. 22A shows sense and antisense guides. FIG. 22B and FIG. 22C show guide screening electropherograms results for sense guides and antisense guides, respectively.

FIG. 23A-FIG. 23C show EGFR L858R guide screening and results using PfAgo. FIG. 23A shows sense and antisense guides. FIG. 23B and FIG. 23C show guide screening electropherograms results for sense guides and antisense guides, respectively.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods that are, for clarity, described herein in the context of separate embodiments, may also be provided in combination in a single embodiment. Conversely, various features of the disclosed methods that are, for brevity, described in the context of a single embodiment, may also be provided separately or in any subcombination.

The disclosure of each patent, patent application, and publication cited or described in this document is incorporated herein by reference, in its entirety.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

As used herein, the term “mutation” refers to any variation in a nucleic acid sequence compared to a wildtype nucleic acid sequence, regardless of the frequency of the mutation. The terms “mutation” and “variation” may be used interchangeably. The terms “mutant” and “variant” may also be used interchangeably.

A person of ordinary skill in the art will understand that the term “low-copy number” or “low-copy” nucleic acid as used herein refers to a species of nucleic acid, for example an allele, a mutant, or a variant of a nucleic acid, that is present in relatively lower proportion than other species of nucleic acid in a population of nucleic acids. That is, the abundance of a low-copy nucleic acid is lower in proportion than the abundance of a non-low-copy nucleic acid in a population of nucleic acids. In one example, a low-copy nucleic acid refers to the fraction or proportion of a mutant allele in a population of nucleic acids containing mutant and non-mutant alleles. The person of ordinary skill will further appreciate that enrichment of a low-copy nucleic acid as referred to herein indicates increasing the proportion or the fraction of the low-copy nucleic acid relative to the population of nucleic acids. The present methods can achieve this result by, for example, first reducing the abundance of non-low copy nucleic acid, thereby increasing the relative abundance of the low-copy nucleic acid, and/or second amplifying the low-copy nucleic acid, thereby further increasing the relative abundance of the low-copy nucleic acid.

Disclosed herein are methods of enriching selected nucleic acids to enhance downstream detection methods. Central to the discriminatory enhancement of a subset of nucleic acids (“target nucleic acids”) that are often low-copy number nucleic acids is the utilization of at least one member of the prokaryotic Argonaute protein (pAgo) family of endonucleases to cleave non-target nucleic acids. These endonucleases, when in the presence of one or more 5′-phosphorylated DNA guides, can specifically bind and cleave non-target nucleic acids, which allows for the relative enrichment of target nucleic acids compared to those cleaved by the pAgo endonuclease. The 5′-phosphorylated DNA guide has a sequence sufficiently complementary to the non-target nucleic acids to allow hybridization of the guide to the non-target nucleic acid. This binding of the guide to the non-target nucleic acid promotes a conformational change in the nucleic acid that activates the Argonaute protein's endonuclease function.

Enrichment of target nucleic acids by cleaving non-target nucleic acids can enhance downstream applications such as amplification and/or sequencing. For example, a sample from a patient can comprise a population of similar nucleic acids, only a few of which contain important clinical information such as mutations associated with certain types of cancer. Detection of these clinically relevant mutations is challenging because mutant alleles are often present at very low concentrations compared to the wild type (WT) nucleic acids. To enhance detection of relatively infrequent mutant alleles of interest by nucleic acid amplification and/or sequencing, it is desirable to reduce the concentration of WT nucleic acids. The enrichment assay can consist of a sample containing a blend of WT DNA and rare mutant alleles, guide DNA complementary to WT-DNA segments, and a DNA cleaving pAgo. The DNA guides hybridize to the complementary segments of the WT-DNA and enable the pAgo to cleave the WT DNA.

One embodiment of the present disclosure provides methods of enriching a target nucleic acid in a sample that comprises contacting the sample with a guide nucleic acid having a sufficiently complementary sequence to a non-target nucleic acid to allow hybridization of the guide nucleic acid and the non-target nucleic acid to form a guide/non-target hybrid, contacting the sample with an endonuclease having an affinity for the guide/non-target hybrid, and amplifying the target nucleic acid. In some aspects, the target nucleic acid is a low-copy nucleic acid and/or the non-target nucleic acid is present in sufficient amounts or concentrations to effectively inhibit the detection of target nucleic acid. For example, when the concentration of non-target nucleic acid present in a sample is greater than that of the target nucleic acid, the non-target nucleic acid will more likely interact with those reagents necessary for amplification or detection compared to the less prevalent target nucleic acid. Such a scenario wherein the non-target nucleic acid is present in excessive amounts or concentrations can occur when the non-target nucleic acid is a wildtype nucleic acid and the target nucleic acid is a low-copy number mutant.

In some aspects, the amount of the target nucleic acid is less than about 10% of the amount of the non-target nucleic acid. In some aspects, the amount of the target nucleic acid is less than about 9%, 8%, 7%, 6%, 5%, 4%, 3%, 2%, 1%, or even less than 1% of the amount of the non-target nucleic acid.

One advantage the methods disclosed herein have over those of the prior art is that the presently disclosed methods can be optimized such that cleavage of non-target nucleic acids and amplification of the target nucleic acid can be performed simultaneously. Using thermophilic endonucleases that have cleavage activity at or near a temperature sufficient for isothermal amplification, sequencing, or other detection reactions allows for simultaneously running the cleavage and detection reactions.

One type of family of thermophilic proteins contemplated in this disclosure is the Argonaute protein family. These proteins are characterized by PAZ (Piwi-Argonaute-Zwille) and P-element Induced Wimpy testis (PIWI) domains and, in combination with guide nucleic acids, participate in gene silencing. Thus, one aspect of methods disclosed herein comprise employing an endonuclease, wherein the endonuclease is an Argonaute enzyme. In some aspects, the endonuclease is a Thermus thermophilus Argonaute (TtAgo). In some aspects, the endonuclease is a Pyrococcus furiosus Argonaute (PfAgo).

TtAgo has advantages over other systems comprising endonucleases that can be programmed to cleave nucleic acids. The best known system is the clustered regularly interspaced short palindromic repeat (CRISPR). When coupled with a single guide RNA, designed to complement targets of interest, the commonly used Cas9 of Streptococcus pyogenes (SpCas9) initially binds to the 3′ NGG protospacer adjacent motif (PAM) site. Next, the crRNA guides base pairs with the protospacer target sequence, positioning the Cas9 nuclease domain to cut the double-stranded DNA three nucleotides upstream of the PAM site. Cas9 can cleave DNA directly. Since the target sequence (outside of the PAM site) can be programmed and multiplexed without any significant off-target effects, Cas9 can deplete specific unwanted high-abundance sequences, enriching rare alleles. TtAgo does not require a PAM site or any other sequence specific motif, and is programmed simply by the hybridization of the guide nucleic acid to the non-target nucleic acids. Thus, in some embodiments, the target nucleic acid does not comprise a protospacer adjacent motif. Some embodiments of the present disclosure provide for methods of enriching a target nucleic acid in a sample that comprising contacting the sample with a guide nucleic acid having a sufficiently complementary sequence to a non-target nucleic acid to allow hybridization of the guide nucleic acid and the non-target nucleic acid to form a guide/non-target hybrid; contacting the sample with an endonuclease having an affinity for the guide/non-target hybrid; and incubating the sample. This method does not require a subsequent amplification step, although amplification can occur subsequent to the incubation step.

When amplification does occur after the cleavage assay, at least an aliquot of the product of the cleavage assay will serve as the template for a separate amplification or other detection assay. In some aspects of the present disclosure, the cleavage and amplification/detection assays are run consecutively, with the product of the cleavage assay serving as the template for the amplification/detection assay.

In some aspects, the cleavage assay and the amplification, sequencing, or other detection assay are combined into a single reaction vessel. For example, contacting a sample comprising target and non-target nucleic acids with TtAgo and a guide nucleic acid having a sufficiently complementary sequence to the non-target nucleic acid will result in degradation of the nontarget nucleic acid. As this degradation reduces the amount of the nontarget nucleic acid in the sample, ratio of the target nucleic acid to nontarget nucleic acid increases.

The reaction conditions for the cleavage assay and the amplification or other downstream assay can also differ. In some aspects, the product of the cleavage assay can be isolated or the buffer used in the cleavage assay can be exchanged for the buffer used in the downstream assay. In some aspects, the TtAgo enzyme can either be removed or deactivated prior to using at least an aliquot of the cleavage assay as the template for the downstream assay. In some aspects, the endonuclease can be removed before amplifying the target nucleic acid. In other aspects, deactivation of the TtAgo enzyme can be temperature dependent or require the addition of a denaturant or other inhibitor of the enzyme.

In some aspects of the present disclosure, amplifying the target nucleic acid comprises polymerase chain reaction (PCR), digital drop PCR, loop-mediated isothermal amplification (LAMP), recombinase polymerase amplification (RPA), or any combination thereof. RAMP is a two stage multiplexed amplification process that combines both LAMP and RPA and is the subject of U.S. Provisional Patent Application No. 62/278,095, “Multiple Stage Isothermal Enzymatic Amplification” and International Patent PCT/US2017/013403, “Multiple Stage Isothermal Enzymatic Amplification.” The present disclosure incorporates by reference each of these applications in their entirety. Amplifying the target nucleic acid can also include, for example, nucleic acid sequence-based amplification (NASBA), self-sustained sequence replication (3SR), rolling circle (RCA), ligase chain reaction (LCR), strand displacement amplification (SDA), multiple displacement amplification (MDA), or helicase-dependent amplification (HDA).

While isothermal amplification can allow the simultaneous cleavage and amplification of non-target and target nucleic acids, respectively, thermocycling methods can also be used when the amplification process is subsequent to the cleavage assay. Thus, some embodiments of the present disclosure provide methods of enriching a target nucleic acid in a sample that comprising contacting the sample with a guide nucleic acid having a sufficiently complementary sequence to a non-target nucleic acid to allow hybridization of the guide nucleic acid and the non-target nucleic acid to form a guide/non-target hybrid; contacting the sample with an endonuclease having an affinity for the guide/non-target hybrid; and incubating the sample. In some embodiments, the amplification of target nucleic acid comprises a polymerase chain reaction (PCR) that uses primers specific for the target nucleic acid.

Because the amount or concentration of target nucleic acid in a sample can be very low compared to the non-target nucleic acid, ddPCR can be used to reduce the probability of false negatives and/or better understand the amount of the target nucleic acid in the sample. ddPCR utilizes microbubbles to encapsulate many small subsamples, and each set of subsamples are then separately amplified. As primers used for specific mutations will only induce amplification of the target nucleic acid, the target nucleic acids in subsamples will be relatively more abundant compared to the non-target nucleic acid and will be amplified.

Detecting rare variants associated with disease in a patient can allow for treatment optimization and significantly improve clinical outcomes. Table 1 shows a non-exhaustive list of mutations associated with cancer. For example, a mutation such as EGFR L858R associated with non-small cell lung cancer (NSCLC) can be targeted with the frontline inhibitor erlotinib, while the mutation EGFR T790M confers resistance to frontline therapy but can be targeted with second and third line inhibitors. While KRAS mutations, detected in the majority of pancreatic cancer tumors, cannot currently be therapeutically targeted, monitoring of the allele fraction of these mutations can serve as a surrogate for solid tumor burden and thus indicate response to therapy.

TABLE 1
Nonexclusive list of mutations associated with cancer

| Gene | Hotspot mutations | | | |
|---|---|---|---|---|
| KRAS | G12R | G12D | G12V | G13D |
| | (34 G > C) | (35 G > A) | (34 G > T) | (38 G > A) |
| EGFR | T790M | L858R | | |
| | (2369 C > T) | (2573 T > G) | | |
| BRAF | V600E | | | |
| | (1799 T > A) | | | |
| PIK3CA | E542K | E545K | H1047R | H1047L |
| | (1624 G > A) | (1633 G > A) | (3140 A > G) | (3140 A > T) |
| NRAS | Q161K | Q61R | | |
| | (181 C > A) | (182 A > G) | | |

Table 1: A sample of hotspot mutations significant to cancer. Only the KRAS region is amenable for enrichment with CRISPR-Cas9 as it includes a Cas9-PAM site. In contrast, all sequences can be enriched with PAM-independent TtAgo.

Some embodiments of the present disclosure provide methods of detecting the presence or absence of cell-free circulating tumor nucleic acids (cf-ctNA) in a sample from a subject, comprising contacting the sample with a guide nucleic acid having a sufficiently complementary sequence to a non-cf-ctNA sufficient to allow hybridization of the guide nucleic acid and the non-cf-ctNA to form a guide/non-cf-ctNA hybrid; contacting the sample with an endonuclease having an affinity for the guide/non-cf-ctNA hybrid under conditions suitable for the endonuclease to cleave the non-cf-ctNA; amplifying the ct-cfNA, if any, in the sample; and detecting the presence or absence of cf-ctNA. The cf-ctNA can be less than about 10% as abundant as the non-cf-ctNA, less than about 9% as abundant as the non-cf-ctNA, less than about 8% as abundant as the non-cf-ctNA, less than about 7% as abundant as the non-cf-ctNA, less than about 6% as abundant as the non-cf-ctNA, less than about 5% as abundant as the non-cf-ctNA, less than about 4% as abundant as the non-cf-ctNA, less than about 3% as abundant as the non-cf-ctNA, less than about 2% as abundant as the non-cf-ctNA, or less than about 1% as abundant as the non-cf-ctNA. In some embodiments, the cf-ctNA is about 0.1% as abundant as the non-cf-ctNA, about 0.2% as abundant as the non-cf-ctNA, about 0.3% as abundant as the non-cf-ctNA, about 0.4% as abundant as the non-cf-ctNA, about 0.5% as abundant as the non-cf-ctNA, about 0.6% as abundant as the non-cf-ctNA, about 0.7% as abundant as the non-cf-ctNA, about 0.8% as abundant as the non-cf-ctNA, about 0.9% as abundant as the non-cf-ctNA, or greater than about 1% as abundant as the non-cf-ctNA. In some embodiments the cf-ctNA is DNA. In some embodiments, the cf-ctNA is RNA.

As explained supra, the cleavage and amplification assays can be run simultaneously or consecutively, with the product of the cleavage assay serving as the template for the detection assay. In some embodiments, amplifying the cf-ctNA comprises isothermal amplification. Because the polymerases used for isothermal amplification can efficiently synthesize nucleic acid at a temperature that TtAgo can efficiently cleave nontarget nucleic acids, these activities can be combined in a single reaction. It is contemplated herein that the detection of cf-ctNA can be concurrent with the amplification of the nucleic acid. Detection of the cf-ctNA can comprise analyzing the amplified nucleic acid with an assay capable of distinguishing cf-ctNA from non-cf-ct-NA. Nucleic acid analysis assays known to those skilled in the art include, but are not limited to, restriction enzyme analysis, sequencing the amplified nucleic acid, fluorescence detection, Southern blot, or a combination thereof.

It is contemplated herein that the presently disclosed methods can also be used in combination with other enrichment strategies. One enrichment strategy involves the use a peptide nucleic acid (PNA) polymerase amplification clamping technique. PNA oligomers with a sequence complementary to WT DNA in the region susceptible to mutations, such as the KRAS region, hybridize to WT DNA during polymerase amplification. Since PNA oligomers do not significantly bind to mutant alleles, polymerase, in the presence of PNA, amplifies mutants with greater efficiency than WT, allowing mutants' detection when their concentrations exceed 0.1% of WT.

In other aspects of the present disclosure, methods described herein can be used to reduce the concentration of one strain of a pathogen in favor of another strain of the pathogen. For example, two strains of a virus can differ only slightly in their sequences, but one strain can be more pathogenic than the other. Due to the similarity in their sequences, both nucleic acids can amplify in a PCR and discrimination of the more pathogenic strain from the less pathogenic one can not be readily apparent based on analysis of the amplified nucleic acids. However, the cleaving assay that is the subject of this invention can be used to cleave the nucleic acid of the less pathogenic virus, thereby enriching the relative concentration of the nucleic acid of the more pathogenic virus. Thus, in some aspects of the present disclosure, the target nucleic acid and the non-target nucleic acid are from different strains of a virus. In some aspects, the pathogen is a virus. In other aspects, the pathogen is bacteria. In other aspects, the pathogen can be any form an infectious agent. In some aspects, the virus having different strains is Zika virus.

The cleaving enzyme can also be used for signal amplification. The guide nucleic acid, attached to a complementary sequence, biotin, or protein (i.e., antibody or antigen) binds or hybridizes to an immobilized captured molecule of interest (DNA, RNA, antigen, or an antibody) in a sandwich assay. The guide DNA activates the cleaving enzyme. Once activated, the cleaving enzyme cleaves proximate quenched nucleic acids (DNA or RNA) with an appropriate sequence. Once cleaved, the previously quenched nucleic acid emits fluorescence that can be detected. A single enzyme can cleave multiple target reporters. The emission intensity is proportional to the concentration of targets of interest and time, enabling signal amplification and quantification. The cleaving process can, alternatively, produce other detectable by-products that can be detected by various means, including non-optical ones such as electrochemical means, including, for example, amperometry, voltammetry, and coulometry.

Another embodiment presently disclosed are methods of detecting a molecule in a sample, comprising contacting the sample with a first antibody having an affinity for a first epitope on the molecule, wherein in the presence of the molecule a target molecule-first antibody complex is formed. This method further comprises contacting the sample with a probe comprising (i) a second antibody having an affinity for a second epitope on the target molecule, (ii) a guide nucleic acid, and (iii) optionally a linker linking the second antibody to the guide nucleic acid, wherein in the presence of the target molecule-first antibody complex form a complex of the probe and the target molecule-first antibody complex is formed. The next step in the method comprises contacting the sample with a target nucleic acid comprising a first portion labeled with a dye, a second portion labeled with a quencher, and a sequence at least partially complementary to a sequence of the guide nucleic acid, wherein in the presence of the detectable complex, the guide nucleic acid of the probe hybridizes to the target nucleic acid to form a guide-target complex. The method also comprises contacting the sample with an endonuclease having an affinity for the guide-target complex and detecting a signal related to the dye.

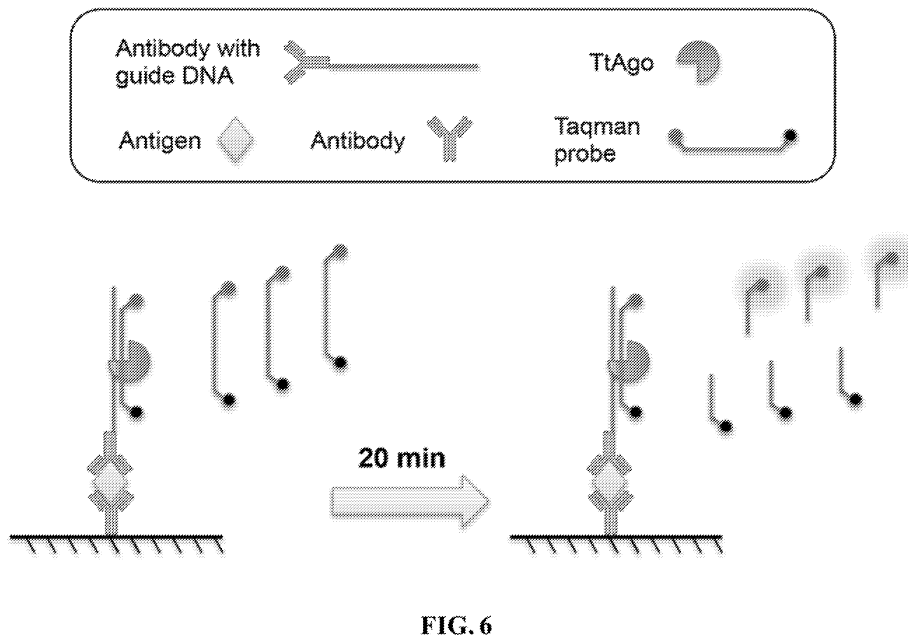

Referring to FIG. 6, a first antibody having specificity for a particular antigen or a first epitope on the antigen is tethered to a substrate. After exposure of the first antibody to a sample comprising the antigen, the antigen and first antibody will form a tethered complex. In some aspects of the present method, an additional step comprises removing molecules not bound by the first antibody. Removing molecules not bound by the first antibody can comprise washing the substrate with a buffer solution or water. A second antibody having specificity for the antigen or a second epitope on the antigen is exposed to the tethered complex. This second antibody has a guide nucleic acid either conjugated directly to the antibody or a linker connected to both the second antibody to the guide nucleic acid. The second antibody will bind to the antigen or second epitope of the antigen to form a detectable complex. Unbound and/or excess second antibody will then be removed. In some aspects, the unbound second antibody is removed in a wash step, wherein saline, water, or other liquid is applied to the substrate and removed via draining, air drying, wicking, or any other method of suitable removing fluid from the substrate.

The guide nucleic acid comprises a sequence having sufficient similarity to a sequence in a probe nucleic acid such that the guide nucleic acid and the probe nucleic acid form a guide-probe complex. Each terminus of the probe nucleic acid is labeled, one termini with a dye and the other with a quencher. Upon exposure to an endonuclease that recognizes the guide-probe complex, the endonuclease will cleave the target nucleic acid, freeing the dye from the quencher and generating a detectable signal. The emission intensity will be proportional to the concentration of probe nucleic acids bound to the guide nucleic acids. Thus, the emission intensity will be proportional to the amount of antigen present in the sample. Some aspects of the method further comprise quantitating the detected signal.

In some embodiments the substrate comprises a microfluidics device, such as, but not limited to, any one of the microfluidic devices disclosed in U.S. application Ser. No. 15/534,810; and International Application No. PCT/US2015/038739. The substrate can also comprise a microchip slide, a resin, or a polymer. In some aspects, the first antibody is tethered to the substrate.

A further aspect of the methods described herein includes enriching a target nucleic acid sequence for next-generation sequencing comprising: protecting, in a population of nucleic acids, a first end of the target nucleic acid with a first pair of inactive Argonaute-guide complex and a second end of the target nucleic acid with a second pair of inactive Argonaute-guide complex; digesting the unprotected nucleic acid with an exonuclease; and detecting the protected nucleic acid. The target nucleic acid can be single stranded or double stranded. The first pair of inactive Argonaute-guide complex can be a first pair of Argonaute proteins complexed with a first pair of DNA guides, and the second pair of inactive Argonaute-guide complex can be a second pair of Argonaute proteins complexed with a second pair of DNA guides.

In some embodiments, the inactive Argonaute-guide complexes comprise an inactivated Argonaute protein, which can be catalytically or enzymatically inactivated, or can be complexed with a guide nucleic acid designed to interfere with the catalytic or enzymatic activity of the Argonaute protein, or both.

The target nucleic acid can be from a pathogen. The population of nucleic acids can be isolated from an organism and the target nucleic acid can comprise a sequence foreign to the organism. Alternatively, the population of nucleic acids can be isolated from an organism and the target nucleic acid can be from a mitochondrial genome of the organism. In some embodiments, the population of nucleic acids can be isolated from a soil sample, a water sample, or a food sample, or the population of nucleic acids can be isolated from a sample from a subject and the target nucleic acid sequence can comprise one or more microbial nucleic acid sequences. Some embodiments further comprise characterizing the microbiome of the subject.

Detecting the protected nucleic acid can comprise hybridization, spectrophotometry, sequencing, electrophoresis, amplification, fluorescence, chromatography, or a combination thereof, or other methods suitable for the detection of nucleic acids.

A further aspect of the methods described herein entails suppressing amplification of non-target nucleic acid by including in a reaction mixture an inactive Argonaute protein-guide complex, wherein the guide is sufficiently complementary to the non-target nucleic acid to form a non-target nucleic acid-inactivated Argonaute protein complex.

### EXAMPLES

The following examples are provided to further describe some of the embodiments disclosed herein. The examples are intended to illustrate, not to limit, the disclosed embodiments.

### Example 1: Enrichment of KRAS G12D Using TtAgo

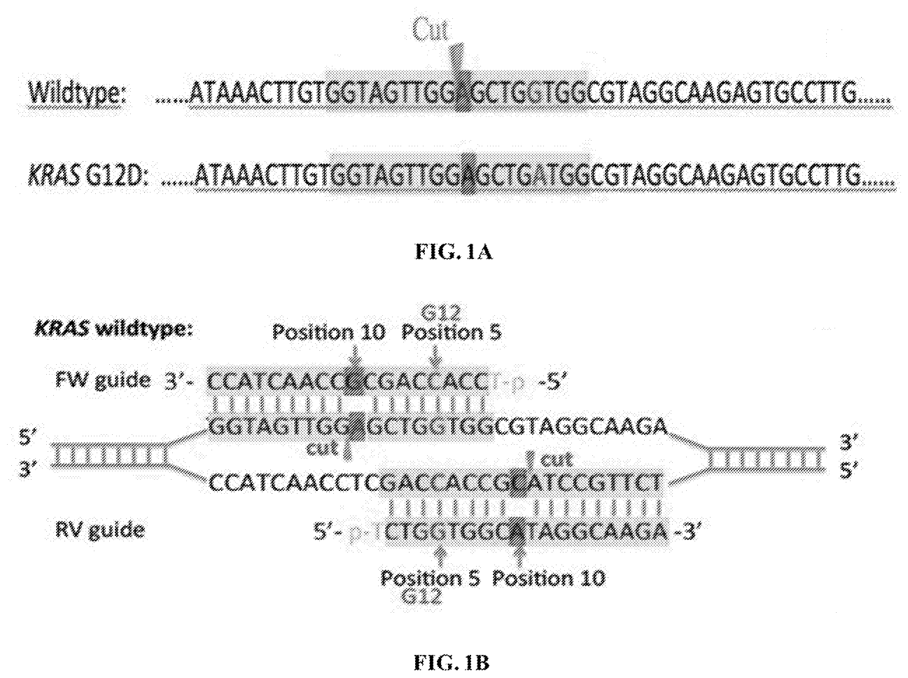

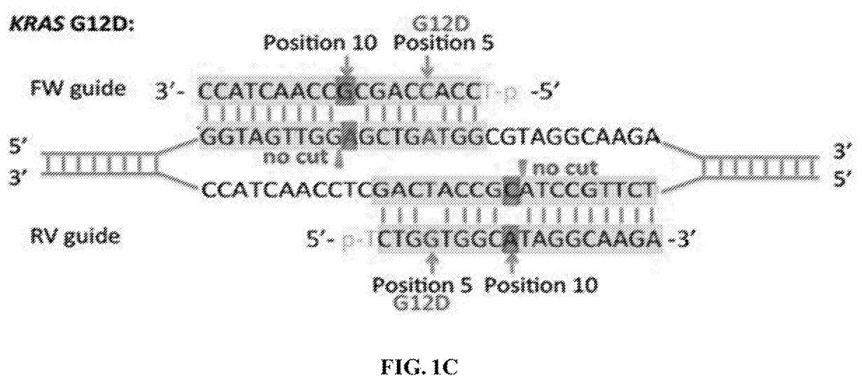

The KRAS G12D mutation is a genetic marker resulting from a single base pair substitution (FIG. 1A). The mutation serves as a negative predictor of radiographic response to the EGFR tyrosine kinase inhibitors administered many aggressive cancers, ranging from lung to pancreatic ductal carcinoma (Misale, Sandra, et al. “Emergence of KRAS mutations and acquired resistance to anti-EGFR therapy in colorectal cancer.” Nature 486.7404 (2012): 532; Eser, S., et al. “Oncogenic KRAS signaling in pancreatic cancer.” British journal of cancer 111.5 (2014): 817.). Early detection of the KRAS G12D mutation, even when the mutation is extremely rare compared to the frequency of the wildtype allele, can result in an optimized treatment strategy and improved clinical outcomes. The mutation can exist in newly transformed cancerous cells and/or in circulating tumor cells that can lead to recurrence of a cancer thought to be in remission or successfully resected.

The presently described methods can surveil low frequency mutations and utilize, for example, TtAgo to cleave wildtype (WT) DNA, while sparing mutant alleles; for example, the presently described methods can utilize TtAgo to cleave wildtype KRAS DNA, while sparing the KRAS G12D mutant. As an example, forward and reverse 5′-phosphorylated single-stranded guide nucleic acids, incorporating a single base-pair mismatch from WT-KRAS to increase specificity, were used to direct TtAgo to the appropriate cut site on WT-KRAS. Because the guide nucleic acid has an additional base pair mismatch at the G12D mutation, it was hypothesized that the guide nucleic acid would not hybridize sufficiently to allow TtAgo to cut the G12A variant. As a general rule, the location of the mismatch can be optimized to maximize its differentiation power, while minimizing its adverse effect on TtAgo cleavage efficiency.

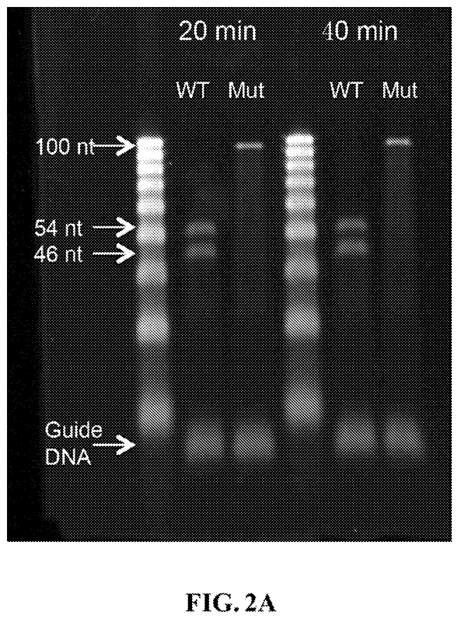

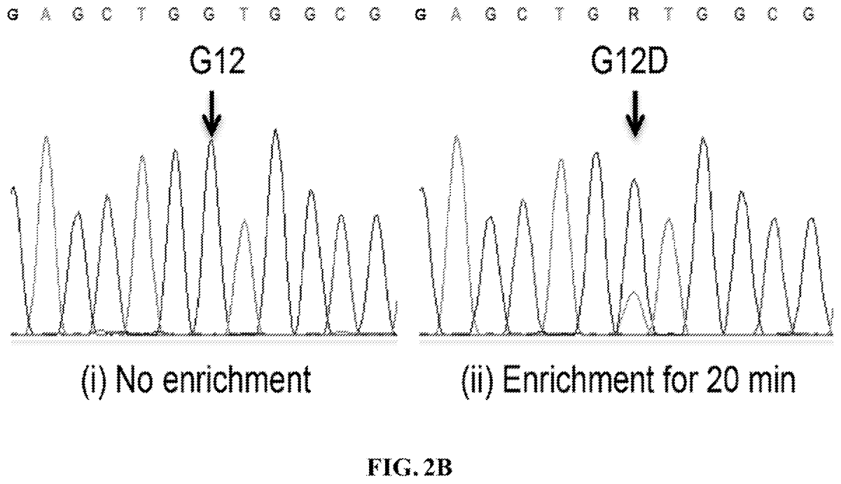

250 nM guide nucleic acid was incubated for 20 or 40 minutes with 250 nM WT-KRAS, 250 nM KRAS G12D, and 1.25 μM TtAgo. Aliquots from each set of reaction conditions were subjected to gel electrophoresis (FIG. 2A). The WT-KRAS lanes exhibit two bands comprising 54 nucleotides (nt) and 46nt, corresponding to the two cleaved segments. The absence of a band at 100nt indicates that most of the WT-KRAS was cleaved. In contrast, the KRAS G12D lanes exhibit single bands at 100nt, corresponding to an intact DNA. TtAgo successfully cleaves WT KRAS DNA while leaving aberrant DNA with a single nucleotide mutation intact.

To illustrate the ability of the disclosed method to effectively enhance the detection of G12D in a sample, a sample that was not incubated with TtAgo was amplified and sequenced. This sequence data shows only a WT-KRAS genotype (FIG. 2B). However, when the sample was incubated with TtAgo prior to amplification, the sequencing data clearly shows the presence of the G12D allele.

This assay can be expanded to interrogate multiple mutations concurrently. The design of guide nucleic acids for a multiplex reaction requires that no guide nucleic acid inhibits another, as this would lead to false negatives and an opportunity for early intervention would be lost. In some aspects, the multiple mutations to be interrogated concurrently are those listed in Table 1. In some aspects, the mutations to be assessed comprise KRAS G12R, G12D, G12V, and G13D; EGFR T790M and L858R; BRAF V600E; PIK3CA E542K, E545K, H1047R, and H1047L; and NRAS Q61K and Q61R.

### Example 2: Enrichment of KRAS G12D Using PfAgo

1 μg of genomic DNA was analyzed without being subjected to a PfAgo-mediated enrichment protocol. The genomic DNA was incubated with dye labeled antibodies capable of binding either nucleic acids carrying the wildtype G12 allele of KRAS or the variant G12D allele and then subjected to flow cytometry to determine the allele frequency of the G12D variant.

To determine if the PfAgo is capable of enriching the variant allele, an additional 1 μg of genomic DNA from the same source was incubated for 20 minutes with PfAgo. The genomic DNA was incubated with dye labeled antibodies capable of binding either nucleic acids carrying the wildtype G12 allele of KRAS or the variant G12D allele and then subjected to flow cytometry to determine the allele frequency of the G12D variant.

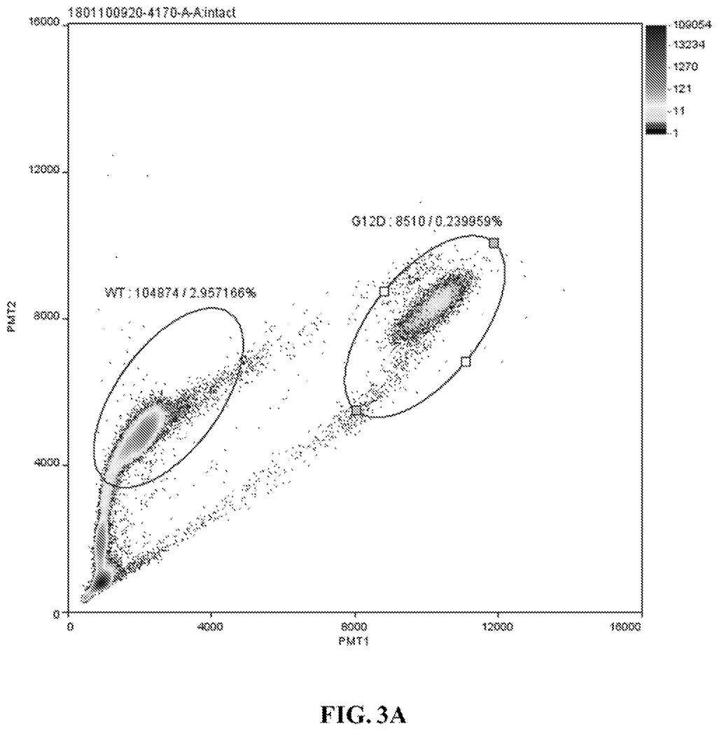

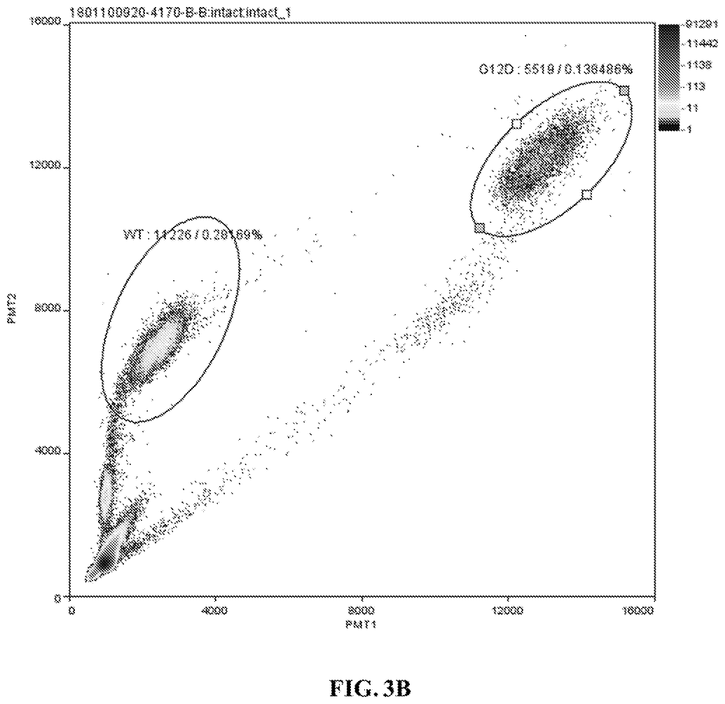

FIG. 3A illustrates that without enrichment the minor variant G12D allele frequency was only 8.5%. In comparison, once the sample was cleaved by PfAgo, the allele frequency of the G12D variant was 49%, a significant increase in the relative amount of the variant allele.

### Example 3: Analysis of Guide Nucleic Sequence and Temperature on PfAgo Cleavage Assay

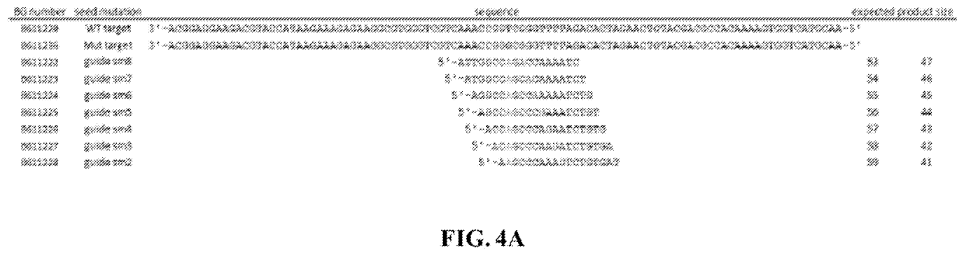

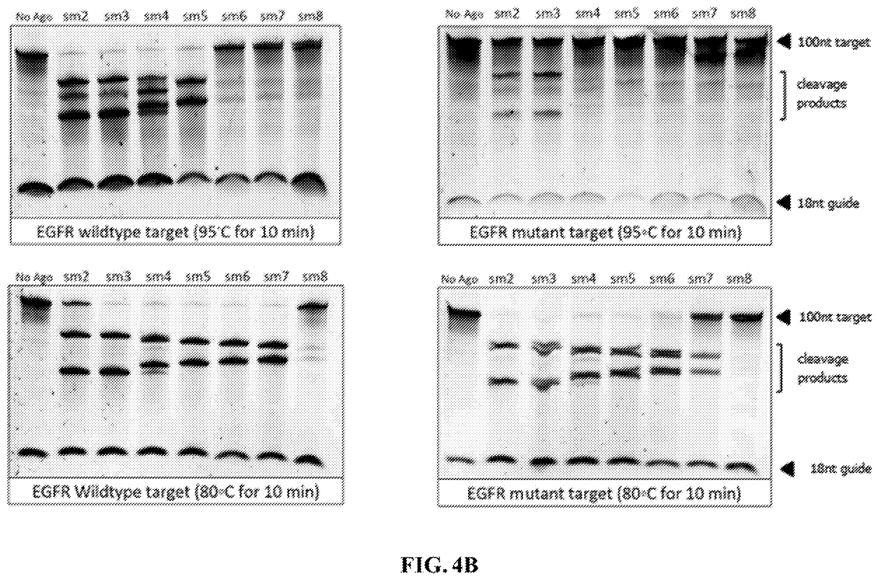

To determine the effect on guide nucleic acid mismatches from wildtype and variant epidermal growth factor receptor (EGFR) nucleic acid, guide nucleic acids comprising 18 nucleotides and a primary mismatch at the mutant locus were constructed. Referring to FIG. 4A, each guide nucleic acid further comprised a different secondary mismatch to test the effect of distance between the primary and secondary mismatches on the cleavage assay. The assays were carried out separately on wildtype and mutant EGFR nucleic acids and at either 80° C. or 95° for 10 minutes.

FIG. 4B presents non-limiting data of the cleavage assay described above. Comparison of the cleavage assays performed at 95° C. shows more cleavage of wildtype samples for every guide nucleic acid used and the proportion of uncleaved nucleic acid was greater in the mutant samples. Additionally, greater distance between the primary and secondary mismatches appears to increase cleavage of the EGFR (both mutant and wildtype) nucleic acid.

The cleavage assays performed at 80° C. for 10 minutes do not show much difference between the wildtype and the mutant EGFR samples. The cleavage profiles for guide nucleic acids sm2-6 are similar for the mutant and wildtype samples, while sm7 and sm8 (i.e., smaller distances between the mismatches) appear to cleave less mutant target nucleic acid. This apparent increase in cleavage could be due to increased annealing of the guide nucleic acids at the lower 80° C. temperature.

### Example 4: Detection of Specific Strains of a Virus

The Zika virus (ZIKV) has two main strains: Asian lineage (the prevailing ZIKV strain in the Americas, called herein ZIKV American strain) and the African lineage. Nucleic acid guides, such as TtAgo, can direct an endonuclease to cleave one virus lineage but not the other, thereby enabling differentiation between these two strains.

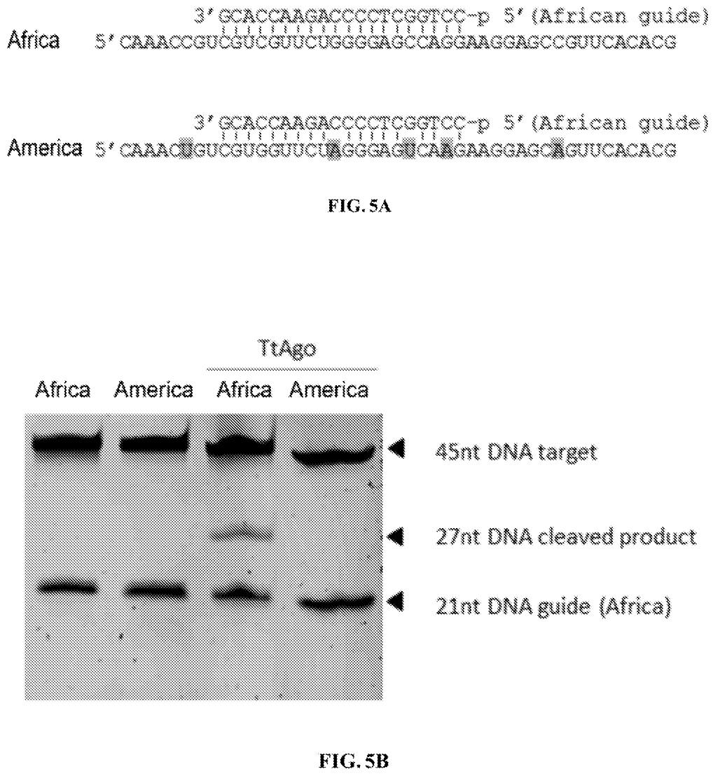

FIG. 5A shows the guide nucleic acid design for differentiating the two different strains of the zika virus. Highly divergent regions between the genomes of these two strains were identified, which allowed design of a guide nucleic acid having three more mismatches with the target strain than the non-target strain. The increased number of mismatches reduced the hybridization rate of the guide nucleic acid with the target strain's RNA, and the non-target zika RNA (or cDNA) was preferably cleaved. FIG. 5B demonstrates that TtAgo is able to discriminate between the different Zika strains. The electrophorograms show that the non-target strain, when incubated with the guide nucleic acid and TtAgo, exhibits two bands while the other strain exhibits only one band. These data indicate that the enzyme cleaved some of the non-target RNA but none of the target RNA. With optimization of the reaction conditions or with guide nucleic acids, more complete cleavage of the non-target RNA can be accomplished.

### Example 5: Optimization of TtAgo Cleavage of Target Nucleic Acids

Betaine, Mg²⁺, and dNTPs Enhance TtAgo's Cleavage Efficiency of Targeted Nucleic Acids

The methods disclosed herein, referred to at times throughout these examples as NAVIGATER (which stands for Nucleic Acid enrichment Via DNA Guided Argonaute from Thermus thermophilus) can be used in combination with enzymatic amplification, in either a single-stage or a two-stage process, for rapid, inexpensive genotyping of rare mutant alleles (MAs). An enzymatic amplification process of particular interest is LAMP since it does not require temperature cycling and can be implemented with simple instrumentation in resource poor settings. We tested TtAgo's activity in three modified variants of LAMP buffers and in a previously described custom cleaving Buffer S (Table 2).

TABLE 2
Buffer Compositions

| | Buffer 2 | | |
|---|---|---|---|
| Buffer 1 | Isothermal | | |
| ThermoPol ® | Amplification | Buffer 3 | Buffer S |
| Reaction | Buffer | Eiken buffer | Buffer of |
| Buffer (1X, NEB) | (1X, NEB) | (1X) | Swarts et.al. |
| 20 mM Tris-HCl | 20 mM Tris-HCl | 20 mM Tris-HCl | 10 mM Tris-HCl |
| 10 mM (NH₄)₂SO₄ | 10 mM (NH₄)₂SO₄ | 10 mM (NH₄)₂SO₄ | 125 mM NaCl |
| 10 mM KCl | 50 mM KCl | 10 mM KCl | 2 mM MgCl₂ |
| 2 mM MgSO₄ | 2 mM MgSO₄ | 8 mM MgSO₄ | (pH 8.0 @ 25° C.) |
| 0.1% Triton ® X-100 | 0.1% Tween ® 20 | 0.1% Tween20 | |
| (pH 8.8 @ 25° C.) | (pH 8.8 @ 25° C.) | 0.8 M Betaine | |
| | | 1.4 mM dNTPs | |
| | | (pH 8.8 @ 25° C.) | |

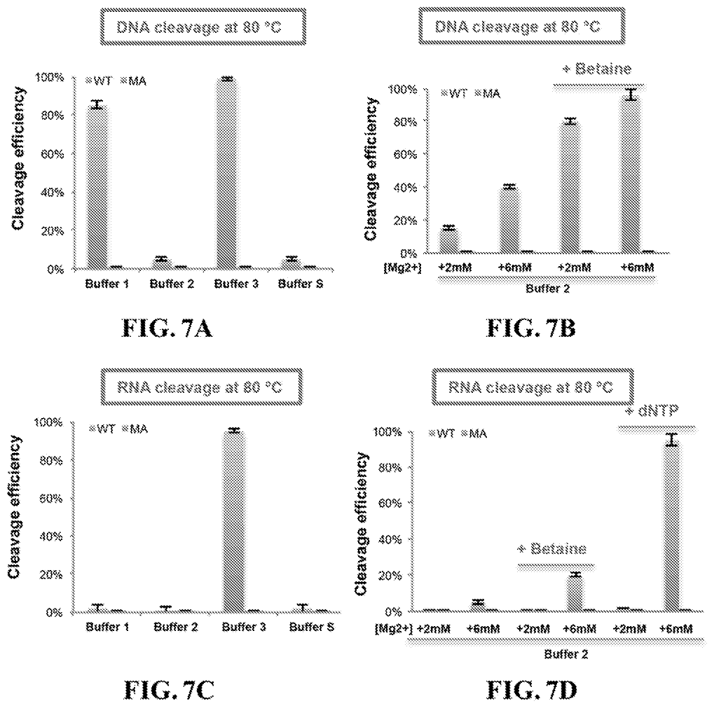

TtAgo was incubated with either ssDNA or ssRNA fragments (100 nt) of the human KRAS gene and a 16 nt guide with a perfect match to the wild type (WT) KRAS, but with a single nucleotide mismatch at guide position 12 (g12) with KRAS-G12D. Cleavage products were subjected to gel electrophoresis. Cleavage efficiency is defined as Γ=I_{C}/(I_{C}+I_{UC}), where I_{C} and I_{UC} are, respectively, band intensities of cleaved and uncleaved alleles. Comparing T in different buffers reveals that the cleavage efficiency in Buffer 3 is nearly 100% for both WT DNA (FIG. 7A) and WT RNA (FIG. 7C) targets, while very low (<1%) for MAs.

Buffer 3's superior performance compared to the other buffers was tested. Unlike the other buffers, Buffer 3 contains betaine, dNTPs, and 4×[Mg²⁺] (8 mM vs 2 mM). To examine the effect of each of these compounds on TtAgo's activity, we varied each additive's concentration in Buffers 2 and S. As the [Mg²⁺] in Buffer S and Buffer 2 increases, so does TWT DNA at 80° C., achieving nearly 100% at [Mg²⁺]˜6 mM (FIG. 7B). Increase in [Mg²⁺] has little effect on TWT DNA at 70° C. (data not shown). TWT RNA increases as [Mg²⁺] increases at both 70° C. and 80° C. (FIG. 7C).

Betaine significantly increases TWT DNA (FIG. 7B) but increases TWT RNA to a lesser degree (FIG. 7D). Supplementing Buffer S with both Mg²⁺ (6 mM) and betaine (0.8 M) increased the TtAgo cleavage efficiency from 60% to nearly 100% (data not shown) at 80° C. This is consistent with betaine's ability to increase thermal stability of polymerase enzymes and to dissolve secondary GC structure during DNA amplification. Addition of 1.4 mM dNTPs increases ΓWT DNA to ˜100% at 80° C. (data not shown) similar to betaine's effect.

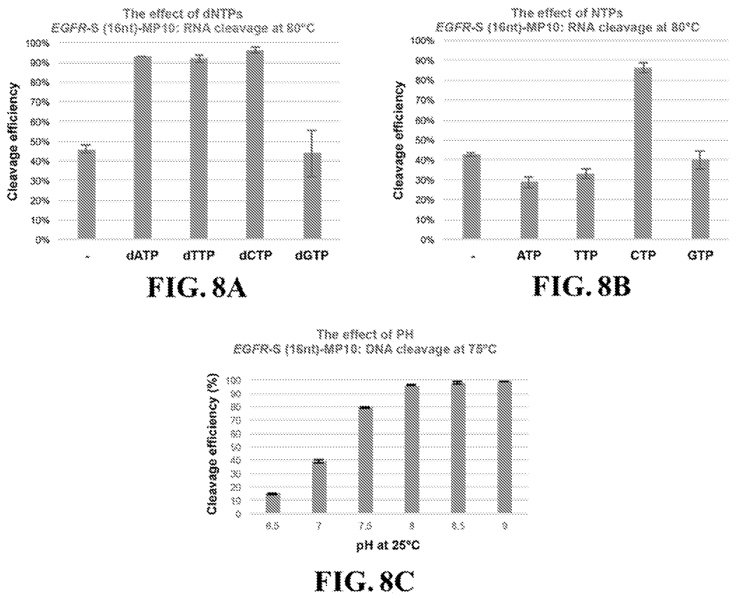

To ascertain that the beneficial effects of dNTPs are not unique to KRAS, we also tested cleavage efficiency of EFGR target sequences. In the absence of dNTPs, EGFR ΓWT RNA ˜45% while in the presence of 1.4 mM dATP, dTTP or dCTP, ΓWT RNA ˜100%. Surprisingly, addition of 1.4 mM dGTP does not affect cleavage efficiency. Among NTPs, only CTP increases ΓWT RNA (FIG. 8B). Although the molecular basis of this phenomenon remains elusive, it appears that the combination of sugar groups and nitrogenous bases of dNTPs stimulates TtAgo's activity.

TtAgo's activity increases as pH increases from 6.6 to 9.0 (FIG. 8C). Among the buffers tested, Buffer 3 provides the best conditions for effective TtAgo cleavage of targeted WT alleles likely due to the presence of betaine, dNTPs, and 8 mM [Mg²⁺]. Notably, TtAgo retains its specificity in the presence of the above additives with ΓMA<1%.

Single Base Pair-Mismatch Discrimination

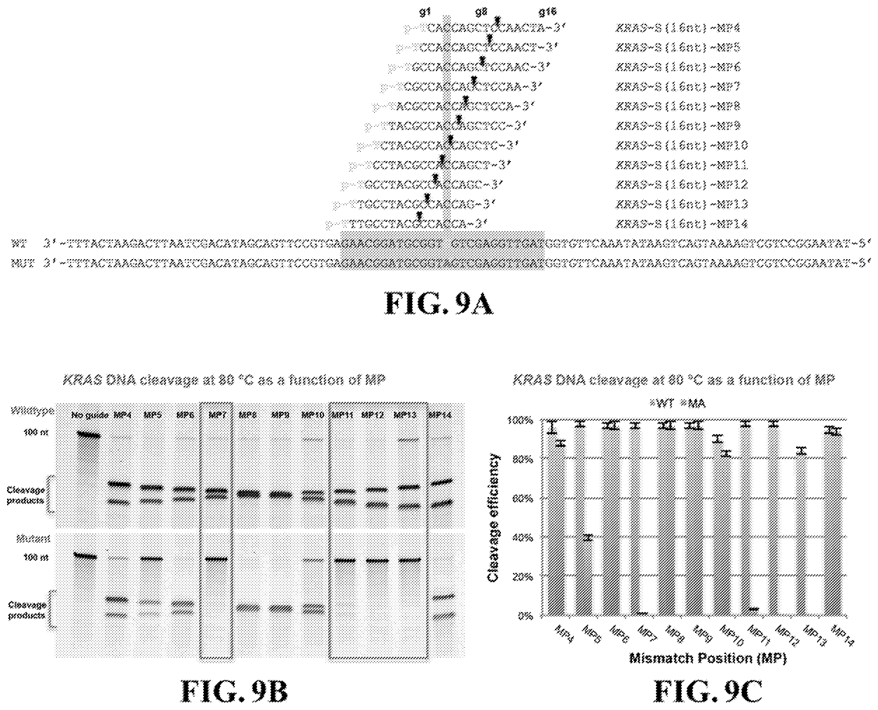

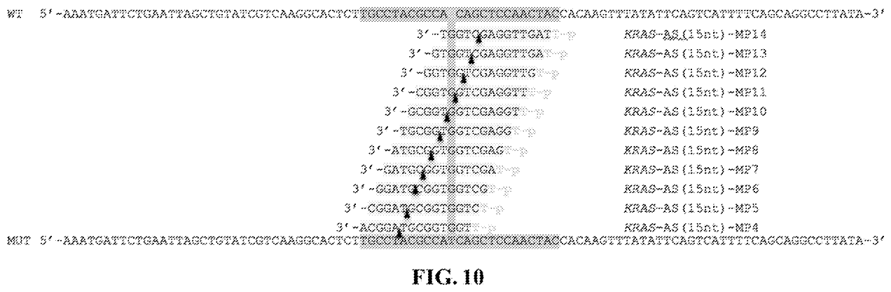

To cleave WT alleles efficiently while sparing alleles with single nucleotide mutations, we designed guide DNAs (gDNAs) with a single base pair mismatch with MAs. A mismatch in the seed region of mouse Argonaute (AGO2) enhances the guide-allele dissociation rate, reducing its cleaving efficiency. Little is known, however, on the effects of guide-allele mismatches on TtAgo's catalytic activity. Molecular dynamic simulations predict that a single DNA guide-mRNA mismatch affects enzyme conformation and reduces activity, but agreement with experiments is imperfect. In the absence of a reliable predictive tool, we analyze experimentally the effect on enzyme activity of a single mismatch type and mismatch position (MP) between guide and KRAS, EGFR, and BRAF sequences (FIG. 9A-9C). We use the notation MP-x to indicate that MA's aberrant nucleotide pairs with guide's x^{th} base, counted from guide's 5′ end. Although all guides are complementary to their targets, variations in MP affected targets' cleavage locations (FIG. 9A), cleavage products' lengths (FIG. 9B), and, unexpectedly, cleaving efficiency (FIG. 9B). For example, antisense (AS) KRAS guide MP-5 and sense (S) EGFR guides MP-7 and MP-12 (FIG. 10) exhibit relatively low cleavage efficiency (data not shown). This suggests that the sequence of the target-guide complex affects enzyme's conformation and activity, even when the guide and target are perfect complements.

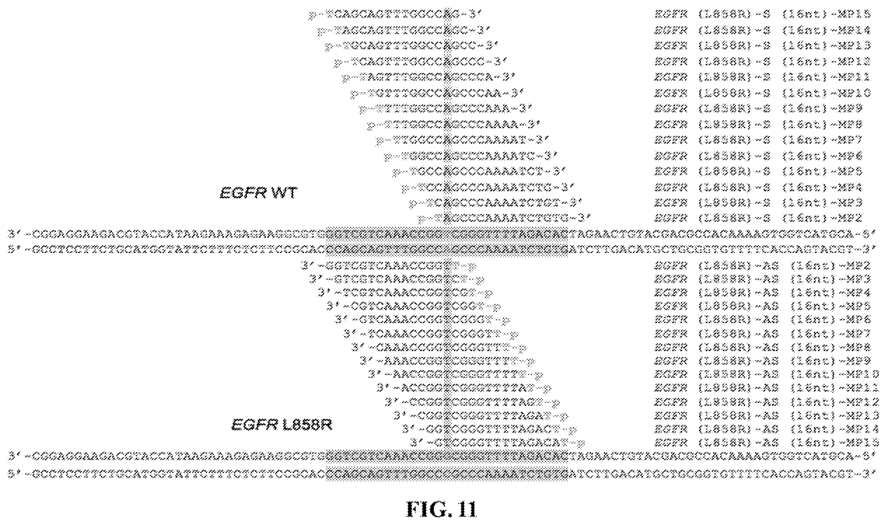

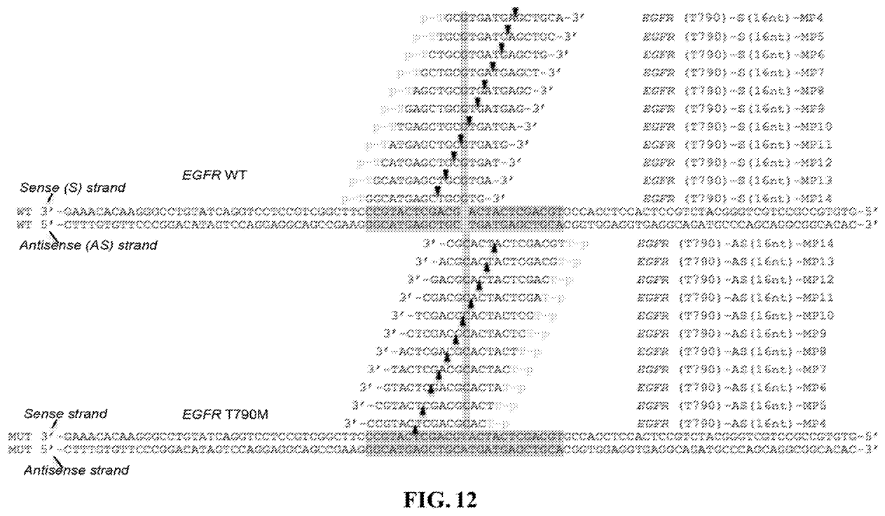

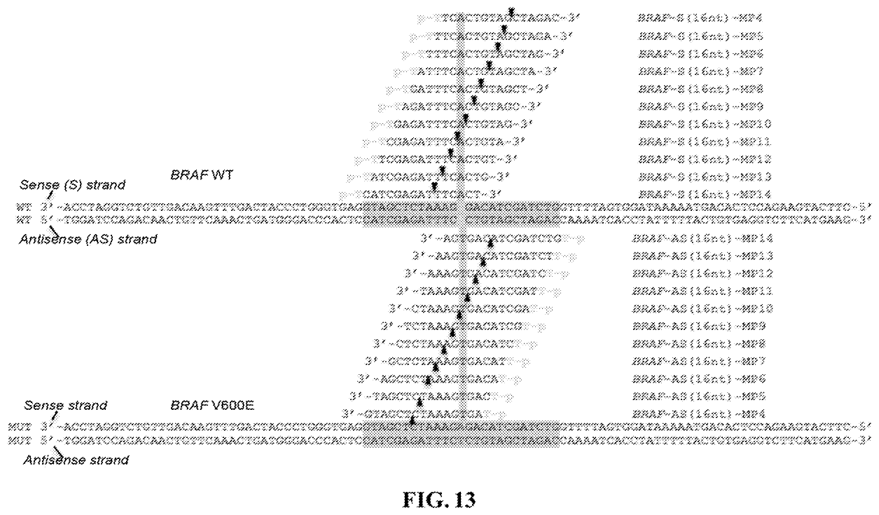

Cleavage suppression of MAs depends sensitively on single base pair mismatch position. Mismatches in both the seed (g2-8) and mid (g9-14) regions diminished cleavage efficiency, and occasionally completely curtailed catalytic activity. For example, KRAS-AS guides (15 nt) MP8-MP14 cleaved KRAS AS G12D (data not shown) and KRAS-S (16 nt) guides MP7 and MP11-MP13 cleaved KRAS S G12D (FIG. 9C) with ΓDNA MA<4%, while MP6 has T DNA MA>40%. The effects of mismatch position on cleaving efficiencies of EGFR WT and L858R, of EGFR T790M, and of BRAF WT and V600E were similarly determined (FIGS. 11, 12, and 13, respectively).

We define discrimination efficiency (DE) as the difference between TtAgo-guide complex cleaving efficiency of WT and that of MA. Mismatches at and around the cleavage site (g10/g11), especially at MP7 and MP9-MP13 yielded the greatest discrimination (DE>80%) for most cases examined (data not shown). The optimal MP depends, however, on the allele's sequence. Cleavage of RNA was more sensitive to MP than cleavage of DNA. Single mismatches at position g4-g11 nearly completely prevented RNA cleavage (data not shown). Our data suggests that guide's sequence differently affects the conformation of the ternary TtAgo-gDNA-DNA and TtAgo-gDNA-RNA complexes.

Short DNA Guides (15/16 nt) Provide Best Discrimination Between WT and MA

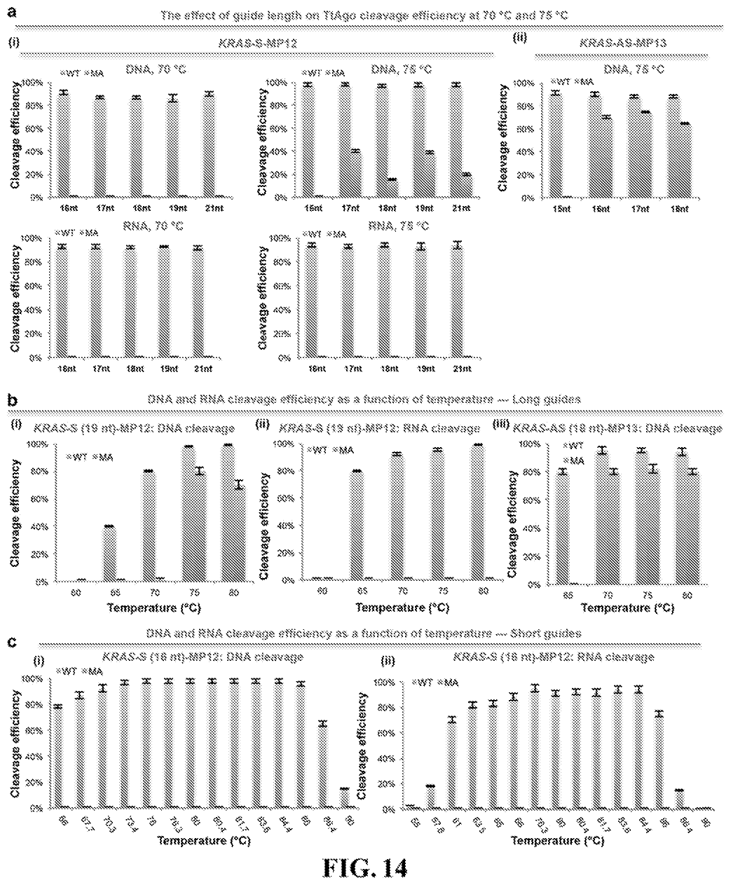

In vitro, TtAgo operates with ssDNA guides ranging in length from 7 to 36 nt. Heterologously-expressed TtAgo is typically purified with DNA guides ranging in length from 13 to 25 nt. Since little is known on the effect of guide's length on TtAgo's discrimination efficiency (DE), we examine the effect of guide's length on DE in our in-vitro assay. TtAgo efficiently cleaves WT KRAS with complementary guides, ranging in length from 16 to 21 nt at both 70° C. and 75° C. (FIG. 14a-i). Guides of 17-21 nt length with a single nucleotide mismatch MP12 cleave MAs at 75° C. but not at 70° C. (FIG. 14a-i, top left). Cleavage of MA at 75° C. is, however, completely suppressed with a short 16 nt guide (FIG. 14a-i). We observe a similar behavior with guides with single mismatches at other positions (data not shown) and with other MA sequences (FIG. 14a-ii). Apparently, TtAgo with shorter guides form a less stable complex with off-targets than longer guides thus preventing undesired cleavage.

In contrast to MA DNA, the increase in temperature did not increase undesired cleavage of MA RNA (FIG. 14a-i, bottom and 4b-ii), likely due to differences in the effects of ssDNA and ssRNA on enzyme conformation. When operating with a short 16 nt guide, single MP12 mismatch, and Buffer 3, TtAgo efficiently cleaves both WT RNA and DNA targets while avoiding cleavage of MAs between 66° C. and 86° C. (FIG. 14c), providing the high specificity that is crucial for an enrichment assay.

TtAgo Efficiently Cleaves Targeted dsDNA Only at Temperatures Above the dsDNA's Melting Temperature

Guide-free TtAgo can degrade dsDNA at low temperatures, and self-generate and selectively load functional DNA guides. This is, however, a slow process that takes place only when target DNA is rich in AT (<17% GC), suggesting that TtAgo lacks helicase activity and depends on dsDNA thermal breathing to enable chopping. Furthermore, since our assay is rich in gDNA that forms a tight complex with TtAgo, TtAgo's direct interactions with dsDNA are suppressed. TtAgo's ability to operate at high temperatures provides the methods described herein with a clear advantage since dsDNA unwinds as the incubation temperature increases.

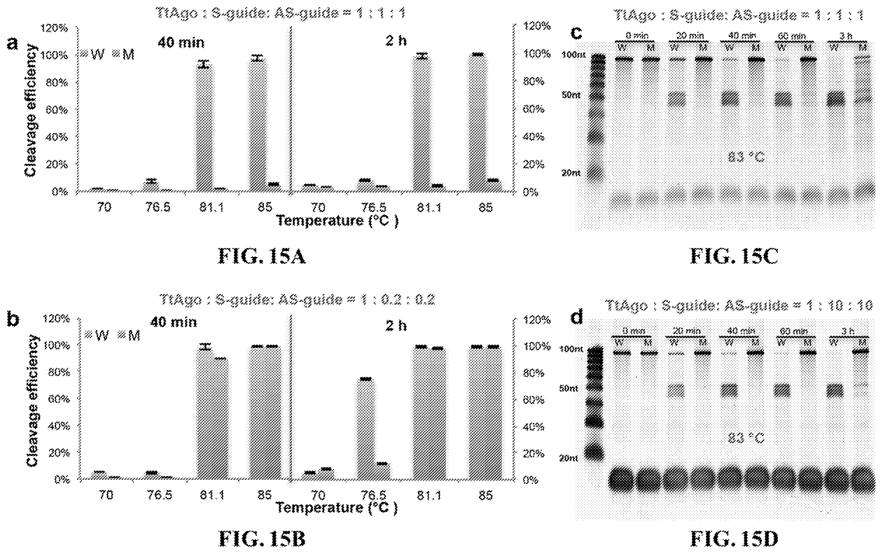

Here, we investigate TtAgo's cleavage efficiency of dsKRAS WT and MA as functions of incubation temperature in the presence of abundant gDNA. The estimated melting temperature of 100 bp dsKRAS (S strand sequence listed in FIG. 9A) in buffer 3 is 79.7° C. (IDT-OligoAnalyzer). Consistent with this estimate, very little cleavage takes place at temperatures under 80° C., but TtAgo cleaves dsDNA efficiently at temperatures above 80° C. (FIG. 15A). Target cleavage efficiency increases as the incubation time increases and saturates after about one hour (FIG. 15C and FIG. 15D). Lengthier incubation time is undesirable as it leads to cleavage of MAs. For efficient discrimination between WT and MA, it is desired to incubate the assay at temperatures exceeding the target melting temperature for less than an hour.

Excess gDNA Concentration is Necessary to Avoid Off-Target Cleavage

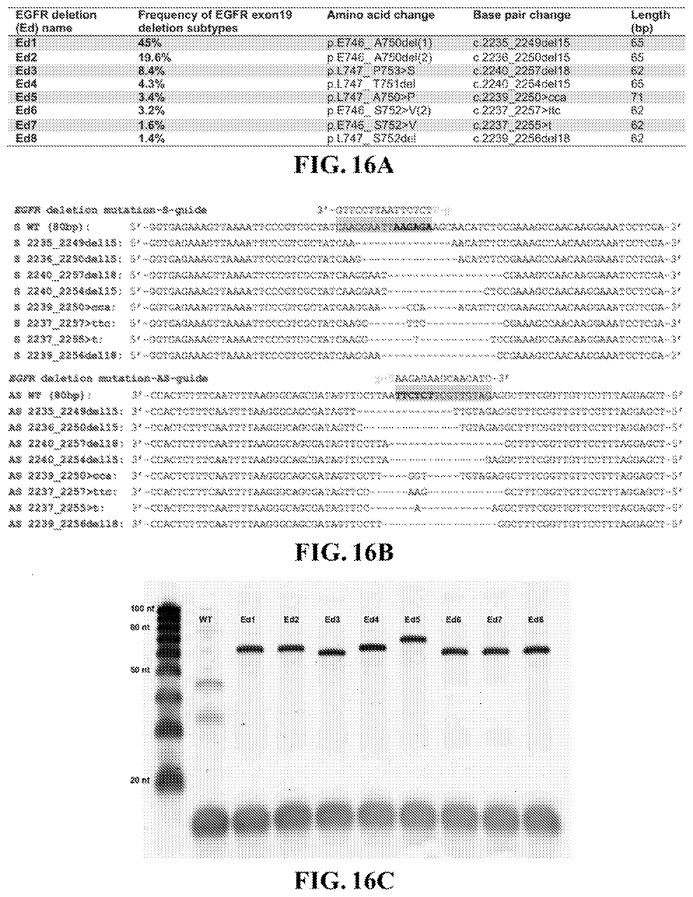

At TtAgo:S-guide:AS-guide ratio of 1:0.2:0.2, non-specific, undesired cleavage of dsMA occurs (FIG. 15B). This off-target cleaving becomes more pronounced as the incubation time increases (FIG. 15B) and can potentially be attributed to TtAgo's self-production of guides from dsDNA (chopping). Since gDNA forms a tight complex with TtAgo, excess gDNA reduces undesired chopping. When guide concentrations exceed TtAgo concentration, no apparent off-target cleavage takes place (FIG. 15C, FIG. 15D). To avoid off-target cleaving, it is necessary to saturate TtAgo with guides and to limit incubation time. Indeed, at TtAgo:S-guide:AS-guide ratio of 1:10:10, the methods described herein efficiently cleave double strand WT KRAS, BRAF and EGFR while sparing point mutations KRAS G12D (FIG. 15), BRAF V600E, and EGFR L858R (data not shown), and EGFR deletion mutations in exon 19 (FIG. 16). In addition, we found the KRAS guides for G12D can also work for discrimination between WT and G12V, and it is enough to form tight TtAgo/guide complex by pre-incubating them on ice for 3 min (data not shown).

### Example 6: Comparison with CRISPR/Cas9-Based dsDNA Cleavage

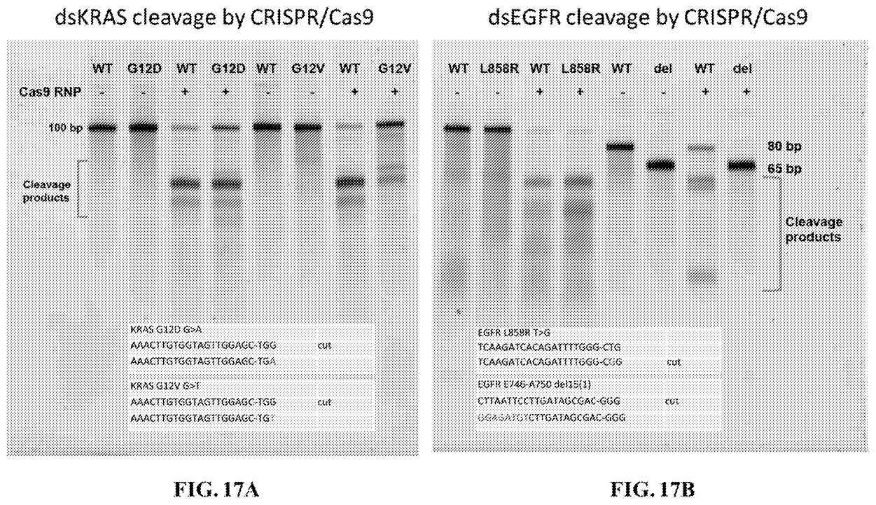

The recently-developed assays DASH and CUT-PCR take advantage of CRISPR/Cas9 low tolerance to mismatches at the PAM recognition site to discriminate between mutant and wild-type alleles. Here, we examine the discrimination efficiency of CRISPR/Cas9 using previously-reported guide RNA (FIG. 17A). CRISPR/Cas9 nonspecifically cleaved both dsWT and dsMAs harboring KRAS G12D and G12V mutations. We suspect that these non-specific cleavages are caused by non-canonical PAM recognition. CRISPR/Cas9 also failed to differentiate between dsWT and dsMA harboring EGFR L858R mutation, presumably because of the presence of a PAM site in EGFR L858R and a non-canonical PAM in the WT (FIG. 17B), which makes it infeasible to design a guide to specifically cleave the WT while sparing the mutant. In contrast, CRISPR/Cas9 specifically cleaved dsWT EGFR while sparing dsMA harboring the deletion mutation E746-A750 del(1). In summary, CRISPR/Cas9 shows lower discrimination efficiency compared with the TtAgo system.

### Example 7: Improving the Sensitivity of Downstream Rare Allele Detection

In recent years, there has been a rapidly increasing interest in applying LB to detect cell-free circulating nucleic acids associated with somatic mutations for, among other things, cancer diagnostics, tumor genotyping, and monitoring susceptibility to targeted therapies. LB is attractive since it is minimally invasive and relatively inexpensive. Detection of MAs is, however, challenging due to their very low concentrations in LB samples among the background of highly abundant WT alleles that differ from MAs by as little as a single nucleotide. To improve detection sensitivity and specificity of detecting rare alleles that contain valuable diagnostic and therapeutic clues, it is necessary to remove and/or suppress the amplification of WT alleles. The methods described herein meet this challenge by selectively and controllably degrading WT alleles in the sample to increase the fraction of MAs. We demonstrate here that single-plex and multiplex methods as described herein increase sensitivity of downstream mutation detection methods such as gel electrophoresis, ddPCR, PNA-PCR, PNA-LAMP, XNA-PCR and Sanger sequencing. Moreover, to demonstrate these methods' potential clinical utility, we enriched blood samples from pancreatic cancer patients, which have been previously analyzed with standard ddPCR protocol (Table 3). These samples were pre-amplified by PCR to increase WT and MA KRAS total content before enrichment.

TABLE 3
The genotype and mutation frequency of 6
samples from pancreatic cancer patients*

| Patient number | P1 | P2 | P3 | P4 | P5 | P6 |
|---|---|---|---|---|---|---|
| Genotype and | NC** | NC** | G12R | G12V | G12D | G12D |
| mutation fraction* | | | 3% | 5% | 0.5% | 20% |

*Samples were analyzed with standard ddPCR protocol.
**Negative control, blood collected from healthy volunteer.
All samples contain similar numbers of WT-KRAS (s.d. <10%).

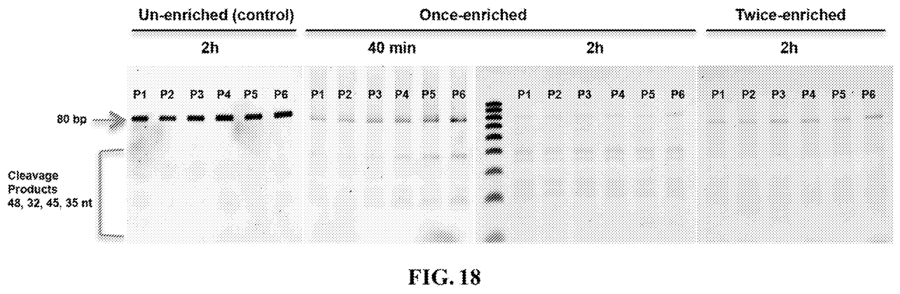

Gel electrophoresis (FIG. 18): We subjected enrichment assay products of pancreatic cancer patients (Table 3) to gel electrophoresis. In the absence of enrichment (control), the bands at 80 bp (KRAS) on the electropherogram are dark. After 40 minutes of TtAgo enrichment, these bands faded, indicating a reduction of KRAS WT alleles. After 2 hours enrichment, all the bands at 80 bp, except that of patient P6, have essentially disappeared, suggesting that most WT alleles have been cleaved. The presence of an 80 bp band in the P6 lane is attributed to the relatively high (20%) MA fraction that is not susceptible to cleaving. We also PCR amplified products from a 2-hour NAVIGATER treatment, and subjected the amplicons to a second NAVIGATER (2 h). The columns P3, P4, and P6 feature darker bands than P1, P2 and P5, indicating the presence of MAs in samples P3, P4, and P6 and demonstrating that NAVIGATER renders observable otherwise undetectable MAs.

Droplet Digital PCR (ddPCR): To quantify our enrichment assay products, we subjected them to ddPCR. The detection limit of ddPCR is controlled by the number of amplifiable nucleic acids in the sample, which must be a small fraction of the number of ddPCR droplets. The large number of WT alleles in the sample limits the number of pre-ddPCR amplification cycles that can be carried out to increase rare alleles' concentration. Since NAVIGATER drastically reduces the number of WT alleles in the sample, it enables one to increase the number of pre-amplification cycles, increasing the number of MAs and ddPCR sensitivity. When operating with a mixture of WT and MA, NAVIGATER products include: residual uncleaved WT (NWT), MA (NMA), and WT-MA hybrids (NH). Hybrid alleles form during re-hybridization of an ssWT with an ssMA. The MA fraction is fMA=(NMA+^{1/2}NH)/(NWT+NMA+NH).

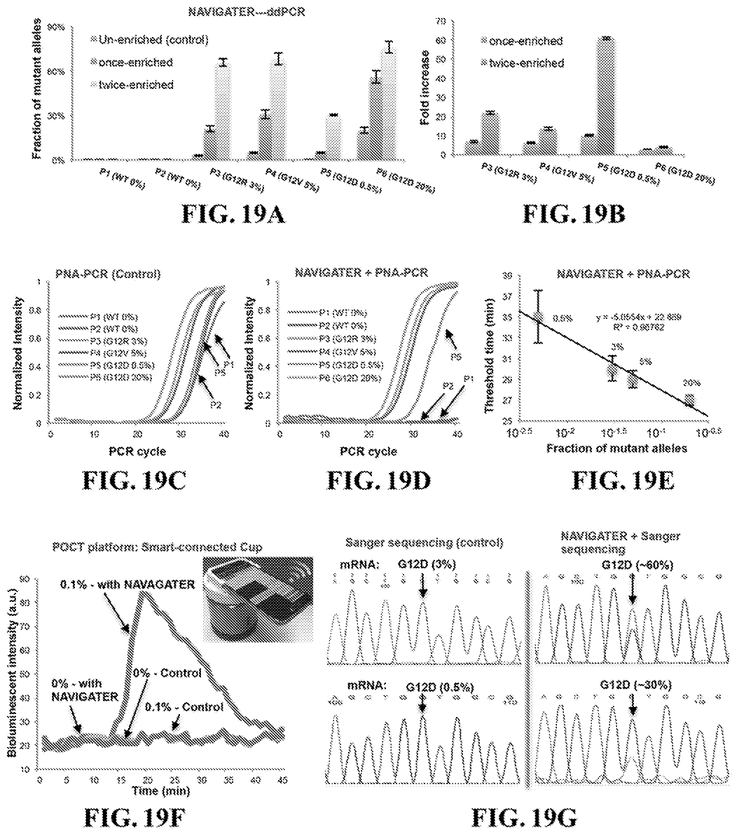

We carried out ddPCR on un-enriched (control, NAVIGATER without TtAgo), once-enriched, and twice-enriched samples, increasing fMA significantly (FIG. 19A). For example, fMA increased from 0.5% in the un-enriched P5 (G12D) sample to ˜30% in the twice-enriched sample. This represents a ˜60 fold increase in the fraction of droplets (fMA) containing MA (FIG. 19B). The same assay also enriched G12R, increasing fMA from 3% to ˜66% in sample P3 and G12V, increasing fMA from 5% to ˜68% in sample P4 (FIG. 19B).

PNA-PCR: PNA-PCR engages a sequence-specific PNA blocker that binds to WT alleles, suppressing WT amplification and providing a limit of detection of fMA ˜1%17. To demonstrate NAVIGATER's utility, we compared the performance of PNA-PCR when processing pancreatic cancer patient samples (Table 3) before and after NAVIGATER (FIG. 19C, FIG. 19D, FIG. 19E). Before enrichment, PNA-PCR real-time amplification curves in the order of appearance are P6, P4, and P3, as expected (Table 3). Samples P1 (fMA=0), P2 (fMA=0), and P5 (fMA=0.5%) nearly overlap, consistent with a detection limit of ˜1%17. Enrichment (FIG. 19D) significantly increases the threshold times of samples P1 and P2, revealing the presence of MAs in sample P5. PNA-PCR combined with NAVIGATER provides the linear relationship T½=22.9−5 log(fMA) between threshold time (the time it takes the amplification curve to reach half its saturation value) and allele concentration (FIG. 19E), allowing one to estimate MA concentration. The data suggests that NAVIGATER can improve PCR-PNA limit of detection to below 0.1%.

PNA-LAMP: Genotyping with PNA blocking oligos can be combined with the isothermal amplification LAMP. To demonstrate the feasibility of genotyping at the point of care and resource-poor settings, we use a minimally-instrumented, electricity-free Smart-Connected Cup (SCC)20 with smartphone and bioluminescent dye-based detection to incubate PNA-LAMP and detect reaction products. To demonstrate that we can also detect RNA alleles, we used simulated samples comprised of mixtures of WT KRAS mRNA and KRAS-G12D mRNA. In the absence of pre-enrichment, SSC is unable to detect the presence of 0.1% KRAS-G12D mRNA whereas with pre-enrichment 0.1% KRAS-G12D mRNA is readily detectable (FIG. 19F).

Sanger Sequencing: In the absence of enrichment, Sanger sequencers detect >5% MA fraction. The Sanger sequencer failed to detect the presence of fMA-3% and 0.5% KRAS-G12D mRNA in our un-enriched samples, but readily detected these MAs following NAVIGATER enrichment (FIG. 19G).

### Example 8: Comparison of TtAgo and CRISPR/Cas9-Based Multiplexed Enrichments Combined with XNA-PCR

We carried out triplex NAVIGATER with 3 different pairs of guides and triplex DASH (a CRISPR/Cas9-based assay8) to enrich samples of 60 ng cfDNA that include WTs and various fractions of KRAS G12D, EGFR ΔE746-A750, and EGFR L858R. The electropherograms results indicate absence of interference among guides, and that both NAVIGATER and DASH are amenable to multiplexing. To evaluate performance of these two enrichment assay, enrichment products were subjected to the clamped assay XNA-PCR that suppresses amplification of WT alleles, enabling detection of MAs down to 0.1% fraction. Without pre-enrichment, XNA-PCR detected down to 0.1% KRAS G12D, 0.1% EGFR ΔE746-A750, and 1% EGFR L858R (data not shown). With NAVIGATER pre-treatment, XNA-PCR sensitivity increased by over 10 folds to 0.01% KRAS G12D, 0.01% EGFR ΔE746-A750, and 0.1% EGFR L858R (data not shown). DASH showed less enrichment for KRAS G12D and EGFR L858R (data not shown), probably due to CRISPR/Cas9's nonspecific cleavage of these two MAs (FIG. 17). In summary, NAVIGATER can operate as a multiplexed assay, enriching multiple MAs; it is more specific than CRISPR/Cas9's PAM site recognition-based enrichment; and it can be combined with XNA-PCR to significantly improve XNA-PCR sensitivity.

### Example 9: Enrichment of Nucleic Acids Using Pyrococcus furiosus Argonaute (PfAgo)

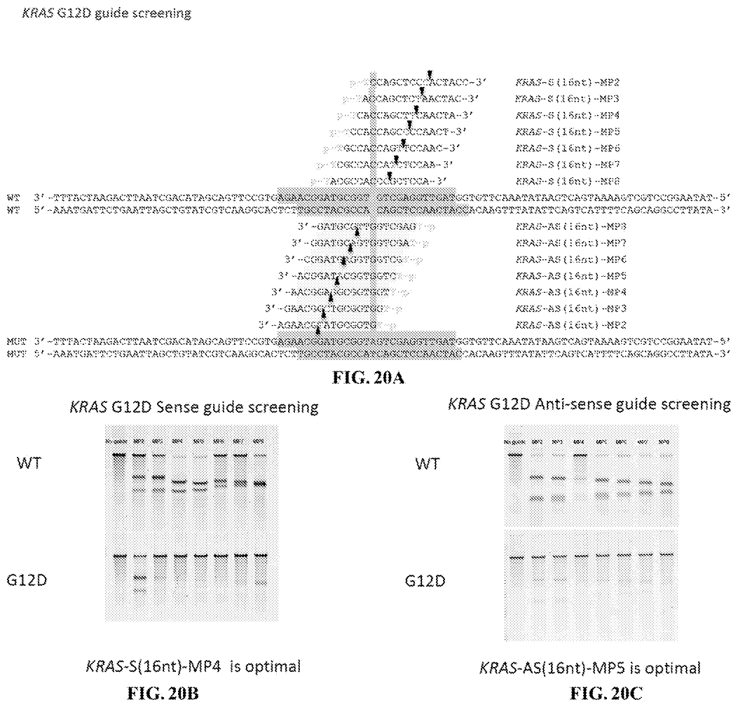

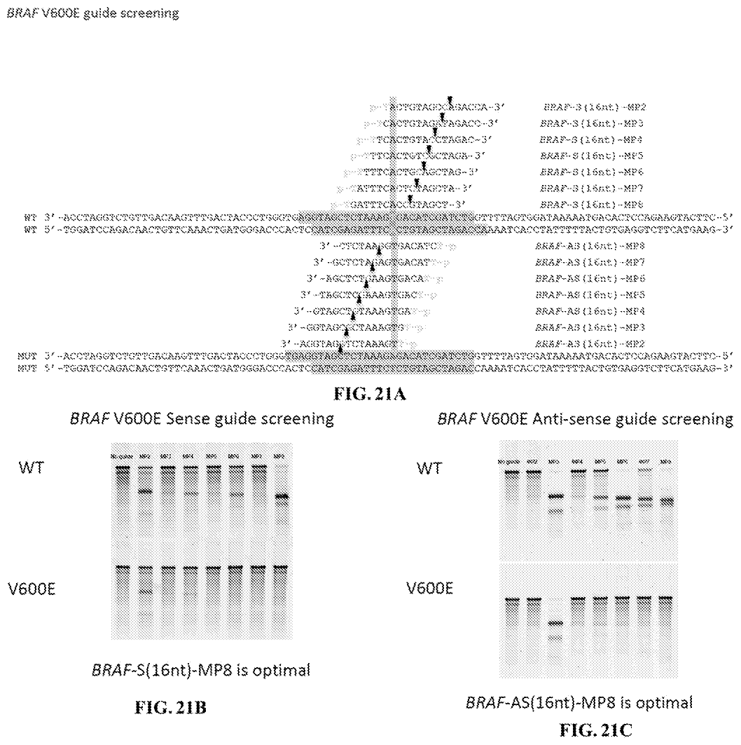

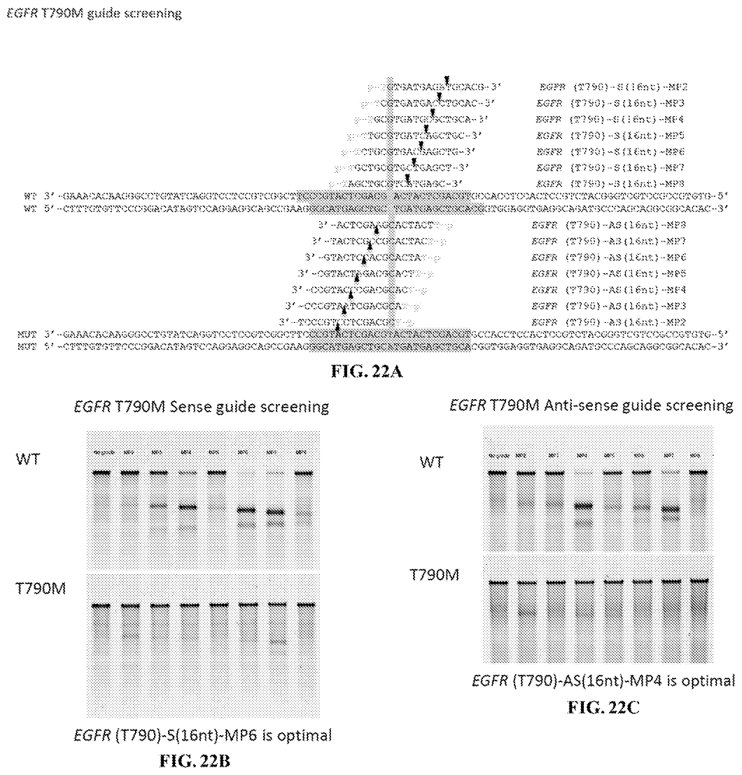

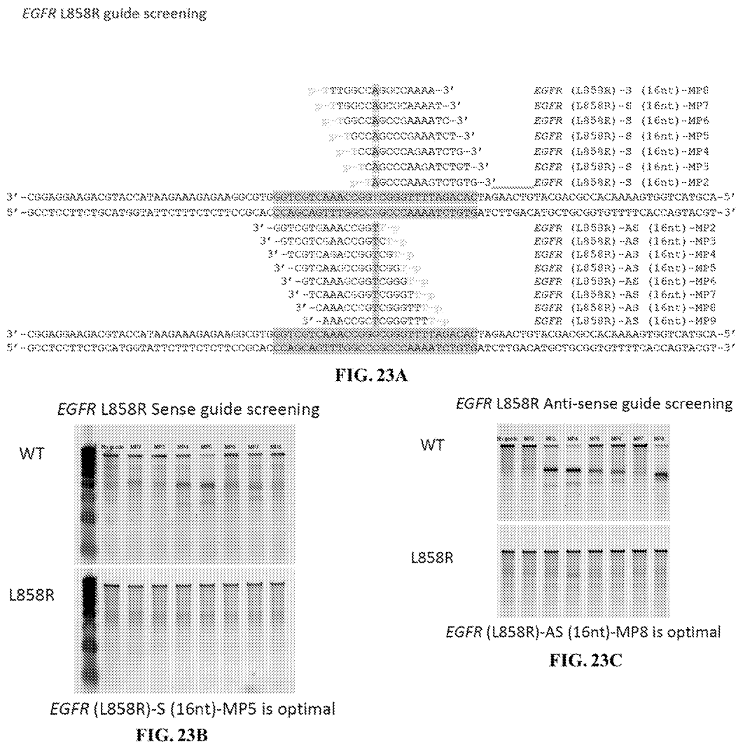

All experiments were carried out with PfAgo at 1.25 μM with an incubation buffer of 15 mM Tris/HCl pH 8, 250 mM NaCl, 0.5 mM MnCl₂, 2.5 μM guide nucleic acid, and 0.25 target nucleic acid. Incubation conditions were 95° C. for 20 minutes. Reaction optimization results for KRAS G12D guide screening are shown in FIG. 20; for BRAF V600E in FIG. 21 ( ), for EGFR T790M in FIG. 22, and for EGFR L858R in FIG. 23.

### Discussion

LB is a simple, minimally invasive, rapidly developing diagnostic method to analyze cell-free nucleic acid fragments in body fluids and obtain critical diagnostic information on patient health and disease status. Currently, LB can help personalize and monitor treatment for patients with advanced cancer, but the sensitivity of available tests is not yet sufficient for patients with early stage disease or for cancer screening. Detection of alleles that contain critical clinical information is challenging since they are present at very low concentrations among abundant background of nucleic acids that differ from alleles of interest by as little as a single nucleotide.

Here, we report on a novel enrichment method (NAVIGATER) for rare alleles that uses TtAgo. TtAgo is programmed with short ssDNA guides to specifically cleave guide-complementary alleles and stringently discriminate against off-targets with a single nucleotide precision. Sequence mismatches between guide and off-targets reduce hybridization affinity and cleavage activity by sterically hindering the formation of a cleavage-compatible state. We observe that TtAgo's activity and discrimination efficiency depend sensitively on the (i) position of the mismatched pair along the guide, (ii) buffer composition, (iii) guide concentration, (iv) guide length, (v) incubation temperature and time, and (vi) target sequence. TtAgo appears to discriminate best between target and off-target in the presence of a mismatch at or around the cleavage site located between guide nucleotides 10 and 11. Optimally, the buffer should contain [Mg²⁺]≥8 mM, 0.8 M betaine, and 1.4 mM dNTPs. The ssDNA guides should be 15-16nt in length with their concentration exceeding TtAgo's concentration; and the incubation temperature should exceed the target dsDNA melting temperature. NAVIGATER is amenable to multiplexing and can concurrently enrich for multiple MAs while operating with different guides.

We demonstrate NAVIGATER's ability to enrich the fraction of cancer biomarkers such as KRAS, BRAF, and EGFR mutants in various samples. For example, NAVIGATER increased KRAS G12D fraction from 0.5% to 30% (60 fold) in a blood sample from a pancreatic cancer patient. The presence of 0.5% KRAS G12D could not be detected with Sanger sequencer or PNA-PCR. However after NAVIGATER pre-processing, both the Sanger sequencer and PNA-PCR readily identified the presence of KRAS G12D. Additionally, NAVIGATER combined with PNA-LAMP detects low fraction (0.1%) mutant RNA alleles and NAVIGATER combined with PNA-LAMP enables genotyping at the point of care and in resource-poor settings. NAVIGATER improves the detection limit of XNA-PCR by more than 10 fold, enabling detection of rare alleles with frequencies as low as 0.01%.

TABLE 4
Comparison of rare allele enrichment methods

| | | | | | | Blocker |
|---|---|---|---|---|---|---|
| | dCas9-based | DASH and | | | | (PNA or |
| | method | Cut-PCR | NAVIGATER | NaME-PrO | COLD-PCR | DNA)-PCR |
| Enzyme | deactivated | CRISPR Cas | TtAgo | DSN | N/A* | N/A |
| | CRISPR Cas9 | proteins | (low cost) | (high cost) | | |
| | (high cost) | (low cost) | | | | |
| Guide/blocker | ~120 nt | ~120 nt | 15/16 nt | 20-25 nt | N/A | PNA (high |
| | sgRNA (high | sgRNA (high | DNA (low | DNA (low | | cost) |
| | cost) | cost) | cost) | cost) | | DNA (low |
| | | | | | | cost) |
| PAM site | Yes | Yes | No | No | No | No |
| requirement | | | | | | |
| Multi-turnover | No | No | Yes | Yes | N/A | N/A |
| enzyme | | | | | | |
| Target | DNA | DNA | DNA & RNA | DNA | DNA | DNA |
| Incubation | ~7 h | 2 h | <1 h | 22 min | ~2 h | ~2 h |
| Tight | No | No | No | Yes | Yes | Yes |
| temperature | (37° C.) | (37° C.) | (66-86° C.) | (98° C., | (thermal | (thermal |
| control | | | | 67° C.) | cycling) | cycling) |
| Fraction | 0.1% with | 0.01% with | 0.01% with | Routinely | 0.1%-0.5% | 0.1-1% |
| detection | allele- | targeted | XNA-PCR | 0.01% with | with | |
| limit | specific | deep | | HRM/Sanger | MALDI-TOF | |
| | qPCR | sequencing | | sequencing | | |
| Enrichment | 5-10 fold | 5-10 fold | 5-10 fold | 5-20 fold | 5-12 fold | N/A |
| level | | | | | | |
| Specificity | Medium | Medium-high | High | Medium | Medium | Medium |
| Multiplexing | Yes | Yes | Yes | Yes | Yes | Yes |
| capability | | | | | | |

*N/A—not applicable

NAVIGATER differs from previously reported rare allele enrichment methods in several important ways (Table 4). First, NAVIGATER is versatile. In contrast to CRISPR-Cas9 and restriction enzymes, TtAgo does not require a PAM motif or a specific recognition site. A gDNA can be designed to direct TtAgo to cleave any desired target. Second, TtAgo is a multi-turnover enzyme; a single TtAgo-guide complex can cleave multiple targets. In contrast, CRISPR-Cas9 is a single turnover nuclease. Third, whereas CRISPR-Cas9 exclusively cleaves DNA, TtAgo cleaves both DNA and RNA targets with single nucleotide precision. Hence, NAVIGATER can enrich for both rare DNA alleles and their associated exosomal RNAs, further increasing assay sensitivity. Fourth, TtAgo is robust, operates over a broad temperature range (66-86° C.) and unlike PCR-based enrichment methods, such as COLD-PCR and blocker-PCR, does not require tight temperature control. Moreover, NAVIGATER can complement PCR-based enrichment methods. Fifth, TtAgo is more specific than thermostable duplex-specific nuclease (DSN). Since DSN non-specifically cleaves all dsDNA, DSN-based assays require tight controls of probe concentration and temperature to avoid non-specific hybridization and cleavage of the rare nucleic acids of interest. Most importantly, as we have demonstrated, NAVIGATER is compatible with many downstream genotyping analysis methods such as ddPCR, PNA-PCR, XNA-PCR, and sequencing. Last but not least, NAVIGATER can operate with isothermal amplification methods such as LAMP, enabling integration of enrichment with genotyping for use in resource poor settings.

### Methods

TtAgo Expression and Purification

TtAgo gene, codon-optimized for E. coli Bl21 (DE3), was inserted into a pET-His6 MBP TEV cloning vector (Addgene plasmid #29656) using ligation-independent cloning. The TtAgo protein was expressed in E. coli Bl21(DE3) Rosetta™ 2 (Novagen). Cultures were grown at 37° C. in Lysogeny broth medium containing 50 μg ml⁻¹ kanamycin and 34 μg ml⁻¹ chloramphenicol until an OD₆₀₀ₙₘ of 0.7 was reached. TtAgo-expression was induced by addition of isopropyl β-D-1-thiogalactopyranoside (IPTG) to a final concentration of 0.1 mM. During the expression, cells were incubated at 18 degrees for 16 hours with continuous shaking. Cells were harvested by centrifugation and lysed in buffer containing 20 mM Tris-HCl pH 7.5, 250 mM NaCl, 5 mM imidazole, supplemented with EDTA-free protease inhibitor cocktail tablet (Roche). The soluble fraction of the lysate was loaded on a nickel column (HisTrap Hp, GE healthcare). The column was extensively washed with buffer containing 20 mM Tris-HCl pH 7.5, 250 mM NaCl and 30 mM imidazole. Bound proteins were eluted by increasing the concentration of imidazole in the wash buffer to 250 mM. The eluted protein was dialysed at 4° C. overnight against 20 mM HEPES pH 7.5, 250 mM KCl, and 1 mM dithiothreitol (DTT) in the presence of 1 mg TEV protease (expressed and purified as previously described) to cleave the His6-MBP tag. Next, the cleaved protein was diluted in 20 mM HEPES pH 7.5 to lower the final salt concentration to 125 mM KCl. The diluted protein was applied to a heparin column (HiTrap Heparin HP, GE Healthcare), washed with 20 mM HEPES pH 7.5, 125 mM KCl and eluted with a linear gradient of 0.125-2 M KCl. Next, the eluted protein was loaded onto a size exclusion column (Superdex 200 16/600 column, GE Healthcare) and eluted with 20 mM HEPES pH 7.5, 500 mM KCl and 1 mM DTT. Purified TtAgo protein was diluted in a size exclusion buffer to a final concentration of 5 μM. Aliquots were flash frozen in liquid nitrogen and stored at −80° C.

TtAgo-Based Cleavage Assays

5′-Phosphorylated DNA guides and Ultramer® ssDNA and ssRNA targets (100 nt) were synthesized by IDT (Coralville, Iowa). For ssDNA and ssRNA cleavage experiments, purified TtAgo, DNA guides, and ssDNA or ssRNA targets were mixed with TtAgo and guides at the ratios indicated in the buffers listed in Table 2 and incubated at the indicated temperatures. Reactions were terminated by adding 1 μL proteinase K (Qiagen, Cat. No. 19131) solution, followed by 15 min incubation at 56° C. Samples were then mixed with 2× loading buffer (95% (de-ionized) formamide, 5 mM EDTA, 0.025% SDS, 0.025% bromophenol blue and 0.025% xylene cyanol) and heated for 10 min at 95° C. before the samples were resolved on 15% denaturing polyacrylamide gels (7M Urea). Gels were stained with SYBR gold Nucleic Acid Gel Stain (Invitrogen) and nucleic acids were visualized using a BioRad Gel Doc XR+ imaging system. For dsDNA cleavage, TtAgo and guides were pre-incubated in LAMP Buffer 3 (Table 2) at 75° C. for 20 min or on ice for 3 min.

CRISPR/Cas9-Based dsDNA Cleavage

Alt-R® S.p. Cas9 Nuclease V3 (Cas9) and Alt-R® CRISPR-Cas9 sgRNA (sgRNA) were purchased from IDT (Coralville, Iowa). To create 10 μM ribonucleoprotein (RNP) complex which contains both sgRNA and Cas9 in equimolar amounts, 10 μM Cas9 and 10 μM sgRNA were incubated in buffer (30 mM HEPES, 150 mM KCl, pH7.5) at room temperature for 10 min. For dsDNA cleavage experiments, RNP complex and dsDNA were mixed in 10:1 ratio (2.5 μM RNP, 0.25 μM dsDNA) in Nuclease Reaction Buffer (20 mM HEPES, 100 mM NaCl, 15 mM MgCl₂, 0.1 mM EDTA, pH6.5) to get 10 μL total volume. The mixture was incubated at 37° C. for 1 h. 1 μL RNase A (Thermo Scientific™, Cat. No. EN0531) was added and incubated at room temperature for 10 min to digest the sgRNA. Then, the Cas9 was digested by adding 1 μL proteinase K (Qiagen, Cat. No. 19131) solution, followed by 15 min incubation at 56° C. Samples were then mixed with 2× loading buffer (95% (de-ionized) formamide, 5 mM EDTA, 0.025% SDS, 0.025% bromophenol blue and 0.025% xylene cyanol) and heated for 10 min at 95° C. before the samples were resolved on 15% denaturing polyacrylamide gels (7M Urea). Gels were stained with SYBR gold Nucleic Acid Gel Stain (Invitrogen) and nucleic acids were visualized using a BioRad Gel Doc XR+ imaging system.

Cell-Free DNA (cfDNA) and RNA Samples

Patient cfDNA samples. All six blood samples (Table 2) were obtained from patients with metastatic pancreatic cancer who had provided informed consent under the IRB-approved protocol (UPCC 02215, IRB #822028). cfDNA was extracted with QIAamp® Circulating Nucleic Acid kit (Qiagen, Valencia, Calif., USA). Subsequently, the extracted cfDNA was qualified and quantified with multiplex ddPCR (Raindance).

RNA samples. Total RNA was extracted with RNeasy® mini kit (Qiagen, Valencia, Calif., USA) per manufacturer's protocol from Human cancer cell lines U87-MG (WT KRAS mRNA) and ASPC1 (KRAS G12D mRNA) and quantified with ddPCR.

cfDNA pre-amplification was carried out in 50-4, reaction volumes using 20 ng of cfDNA, 1×Q5 Hot Start High-Fidelity Master Mix (New England Biolabs, Ipswich, Mass.), and 100 nM each of forward and reverse KRAS 80 bp-PCR primers (Table 5).

TABLE 5
The sequences and concentrations of KRAS
primers, PNA clamp oligo, and Taqman probes
used in downstream mutation analysis.

| | | | Amplicons |
|---|---|---|---|
| | | | length |
| | | | and |
| | | | melting |
| | Sequences | Concen- | tempera- |
| Name | (5′---3′) | tration | ture |
| 80 bp- | AGGCCTGCTGAAA | | 80 bp, |
| PCR-FW | ATGACTGAATAT | | 79.5° C. |
| 80 bp- | GCTGTATCGTC | | |
| PCR-RV | AAGGCACTCTT | | |
| G12-WT- | TTGGAGCTG | 100 nM | |
| VIC probe | GTGGCGT | | |
| G12D-FAM | TGGAGCTGA | 100 nM | |
| probe | TGGCGT | | |
| G12R-FAM | TTGGAGCTCG | 100 nM | |
| probe | TGGCGT | | |
| G12V-FAM | ACGCCAACAGCTC | 100 nM | |
| probe | | | |
| 295 bp- | AAGGTACTGGT | 100 nM | 295 bp, |
| PCR-FW | GGAGTATTTG | | 82.6° C. |
| 295 bp- | GTACTCATGAA | 100 nM | |
| PCR-RV | AATGGTCAGAG | | |
| SMAP-2- | TATTATAAG | 0.4 μM | 123 bp |
| OPI | GCCTGCTG | | |
| SMAP-2- | TTGGATCAT | 0.4 μM | |
| OP2 | ATTCGTCC | | |
| SMAP-2- | ACCTTCTACCCTCA | 3.0 μM | |
| FP | GAAGGTATAAACTT | | |
| | GTGGTAGTTGGAGC | | |
| SMAP-2- | GCAAGAGTGCCTTGA | 1.5 μM | |

BP

| | | |
|---|---|---|
| SMAP-2- | TGGCGTAGGCATGAT | 3.0 μM |
| TP | TCTGAATTAGCTGTAT | |
| PNA clamp | CCTACGCCA | 0.7 μM |
| | CCAGCTCC | |

Table 5 discloses SEQ ID NOS 165-178,
respectively, in order of appearance.

Reaction mixes without DNA were included as no-template (negative) controls (NTCs). Nucleic acids were preamplified with a BioRad Thermal Cycler (BioRad, Model CFD3240) with a temperature profile of 98° C. for 3 minutes, followed by 30 cycles of amplification (98° C. for 10 seconds, 63° C. for 3 minutes, and 72° C. for 30 seconds), and a final 72° C. extension for 2 minutes. RNA pre-amplification was performed in 50-4, reactions using 30 ng of total RNA, 1×Q5 Hot Start High-Fidelity Master Mix (New England Biolabs, Ipswich, Mass.), 100 nM each of forward and reverse KRAS 295 bp-PCR primers (Table 5), and 1 μL reverse transcriptase (Invitrogen, Carlsbad, Calif.). The reaction mix was incubated at 55° C. for 30 minutes and 98° C. for 3 minutes, followed by 30 cycles of amplification (93° C. for 15 seconds, 62° C. for 30 seconds, and 72° C. for 30 seconds), and a final 72° C. extension for 4 minutes.

Mutation Enrichment (NAVIGATER)

The same setup as for synthetic dsDNA cleavage was used for cf-ctDNA and mutant mRNA enrichment. TtAgo, S-guide, and AS-guide were mixed in 1:10:10 ratio (1.25 μM TtAgo, 12.5 μM S-guide, 12.5 μM AS-guide) in the Buffer 3 and pre-incubated at 75° C. for 20 min. Samples consisted of 2 μL preamplified PCR or RT-PCR products were added after pre-incubation of TtAgo and guides. The reaction mixes were incubated at 83° C. for 1 hour. The enriched products were diluted 10⁴ fold before downstream mutation analysis or second-round enrichment. For second-round enrichment, the protocol outlined above was repeated.

NAVIGATER Combined with Downstream Mutation Detection Methods

Droplet digital PCR (ddPCR). ddPCR was carried out with the RainDrop Digital PCR system (RainDance Technologies, Inc.) to verify mutation abundance before and after TtAgo enrichment. 2 μL of the 10⁴-fold diluted, TtAgo-treated sample was added to each 30-μL dPCR. dPCRs contained 1×TaqMan Genotyping Master Mix (Life Technologies), 400 nM KRAS 80 bp-PCR primers, 100 nM KRAS wild-type target probe, 100 nM KRAS mutant target probe (Table 5), and 1× droplet stabilizer (RainDance Technologies, Inc.). Emulsions of each reaction were prepared on the RainDrop Source instrument (RainDance Technologies, Inc.) to produce 2 to 7 million, 5-pL-volume droplets per 25-μL reaction volume. Thereafter, the emulsions were placed in a thermal cycler to amplify the target and generate signal. The temperature profile for amplification consisted of an activation step at 95° C. for 10 minutes, followed by 45 cycles of amplification [95° C. for 15 seconds and 60° C. for 45 seconds]. Reaction products were kept at 4° C. before placing them on the RainDrop Sense instrument (RainDance Technologies, Inc.) for signal detection. RainDrop Analyst (RainDance Technologies, Inc.) was used to determine positive signals for each allele type. Gates were applied to regions of clustered droplets to define positive hits for each allele, according to the manufacturer's instructions.

PNA-PCR. PNA-PCR was performed in 20-μL reaction volumes, containing 4.5 μL of the 10⁴-fold diluted TtAgo-treated products, 1×Q5 Hot Start High-Fidelity Master Mix (New England Biolabs, Ipswich, Mass.), 0.5 μL of EvaGreen fluorescent dye (Biotium, Hayward, Calif.), 500 nM KRAS PNA clamp (Table 5), and 100 nM each of forward and reverse KRAS 80 bp-PCR primers. Reactions were amplified with a BioRad Thermal Cycler (BioRad, Model CFD3240) with a temperature profile of 98° C. for 3 minutes, followed by 40 cycles of amplification (98° C. for 10 seconds, 63° C. for 3 minutes, and 72° C. for 30 seconds).

Sanger sequencing. RNA extracted from cell lines were pre-amplified by KRAS 295 bp-PCR primers as described above and treated by TtAgo mutation enrichment system. 2 μL of the 10⁴-fold diluted, TtAgo-treated sample was amplified by 295 bp PCR protocol (the same as 295 bp RT-PCR protocol without a reverse transcription step) for 30 cycles. PCR products were checked for quality and yield by running 5 μl in 2.2% agarose Lonza FlashGel DNA Cassette and processed for Sanger sequencing at Penn Genomic Analysis Core.

POC mutation detection. PNA-LAMP (SMAP-2) was prepared in 20-μL reaction volumes according to previously described protocol. The reaction mix contained 2 μL of the 10⁴-fold diluted TtAgo-treated products (same as used for Sanger sequencing), 1×LAMP buffer 3 (Eiken LAMP buffer), 1 μL Bst DNA polymerase (from Eiken DNA LAMP kit), 2.5 μL of BART reporter (Lot: 1434201; ERBA Molecular, UK), KRAS PNA clamp and LAMP primers (sequences and concentrations listed in Table 5). The prepared reaction mixtures were injected into reaction chambers of our custom made multifunctional chip. The inlet and outlet ports were then sealed with transparent tape (3M, Scotch brand cellophane tape, St. Paul, Minn.) and the chip was placed in our portable Smart-Connected Cup and processed according to previously described protocol.

Comparison of TtAgo and CRISPR/Cas9-Based Multiplexed Enrichment by Combining them with XNA-PCR

Multiplexed pre-amplification: Triplex PCR were carried out with mutation detection kit (DiaCarta, Inc). The 10-μL reaction mixture contains 60 ng of cfDNA (reference standard that includes various MAs, Horizon Discovery, HD780), 1×PCR Master Mix, 1 μL of either single or mixed PCR primers (1:1:1) for targets of interest. Nucleic acids were pre-amplified with a BioRad Thermal Cycler (BioRad, Model CFX96) with a temperature profile of 95° C. for 5 minutes, followed by 35 cycles of amplification (95° C. for 20 seconds, 70° C. for 40 seconds, 60° C. for 30 seconds, and 72° C. for 30 seconds), and a final 72° C. extension for 2 minutes.

Multiplexed enrichment: For NEVIGATER, guides (1:1:1) for targets of interest were mixed with TtAgo in 10:1 ratio (12.5 μM S-guides, 12.5 μM AS-guides, 1.25 μM TtAgo) in Buffer 3 and pre-incubated on ice for 3 min. Samples consisted of 1 μL pre-amplified triplex PCR products mixed with pre-incubated TtAgo-guide complexes. The reaction mixes were incubated at 83° C. for 1 hour. For DASH, the procedure is the same to the synthetic dsDNA cleavage except using pre-amplified triplex PCR products. The products with and without treatment were resolved on 15% denaturing polyacrylamide gels (7M Urea). The products were diluted 10⁵˜10⁷ fold before downstream mutation analysis.

XNA-PCR: NAVIGATER products were tested by mutation detection method XNA-PCR (DiaCarta, Inc.). XNA-PCR was carried out for individual mutants in 10-μL reaction volumes, containing 3 μL of the 10⁵˜10⁷-fold diluted NAVIGATER products, 1×PCR Master Mix, 1 μL of PCR primer/probe mix, and 1 μL of XNA clamp. Reactions were amplified with a BioRad Thermal Cycler (BioRad, Model CFX96) with a temperature profile of 95° C. for 5 minutes, followed by 45 cycles of amplification (95° C. for 20 seconds, 70° C. for 40 seconds, 60° C. for 30 seconds, and 72° C. for 30 seconds).

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of enriching a target nucleic acid in a sample, comprising:
a. contacting the sample with a guide nucleic acid having a sufficiently complementary sequence to a non-target nucleic acid to allow hybridization of the guide nucleic acid and the non-target nucleic acid to form a guide/non-target hybrid;
b. contacting the sample with an endonuclease having an affinity for the guide/non-target hybrid; and
c. amplifying the target nucleic acid or incubating the sample.

2. (canceled)

3. (canceled)

4. The method of claim 1, wherein the target nucleic acid is a low-copy nucleic acid.

5. The method of claim 1, wherein the target nucleic acid is less than about 10% as abundant as the non-target nucleic acid.

6. The method of claim 1, wherein the target nucleic acid is less than about 0.1% as abundant as the non-target nucleic acid.

7. The method of claim 1, further comprising removing the endonuclease before amplifying the target nucleic acid.

8. (canceled)

9. The method of claim 1, wherein amplifying the target nucleic acid comprises polymerase chain reaction (PCR), digital drop PCR, loop-mediated isothermal amplification (LAMP), recombinase polymerase amplification (RPA), or any combination thereof.

10. The method of claim 1, wherein the target nucleic acid comprises a mutation or is a variant associated with a disease.

11. The method of claim 1, wherein the target nucleic acid and the non-target nucleic acid are from different strains of a pathogen.

12. The method of claim 1, wherein the endonuclease is an Argonaute enzyme, a Thermus thermophiles Argonaute enzyme (TtAgo), or a Pyrococcus furiosus Argonaute enzyme (PfAgo).

13. (canceled)

14. (canceled)

15. The method of claim 1, wherein the guide nucleic acid is DNA.

16. The method of claim 1, wherein the guide nucleic acid is incubated with the endonuclease prior to incubation with the target and non-target nucleic acids.

17. The method of claim 1, wherein the target nucleic acid does not comprise a protospacer adjacent motif.

18. (canceled)

19. The method of claim 1, wherein the target nucleic acid is a DNA or a RNA.

20. (canceled)

21. The method of claim 1, wherein the amplifying comprises employing capping oligos to discourage amplification of non-target nucleic acids.

22. The method of claim 21, wherein the capping oligos are PNA or XNA.

23. (canceled)

24. The method of claim 1, further comprising repeating the contacting and amplifying or incubating steps.

25. A method of detecting the presence or absence of cell-free circulating tumor nucleic acid (cf-ctNA) in a sample from a subject, comprising:
a. contacting the sample with a guide nucleic acid having a sufficiently complementary sequence to a non-target wildtype or NA to allow hybridization of the guide nucleic acid and the non-target wildtype NA to form a guide/non-target or wildtype NA hybrid;
b. contacting the sample with an endonuclease having an affinity for the guide/non-target or wildtype NA hybrid under conditions suitable for the endonuclease to cleave the non-target or wildtype NA;
c. amplifying the ct-cfNA, if any, in the sample; and
d. detecting the presence or absence of cf-ctNA.

26. The method of claim 25, wherein the cf-ctNA is less than about 10% as abundant as the non-cf-ctNA.

27. The method of claim 25, wherein the cf-ctNA is less than about 0.1% as abundant as the non-target or wildtype NA.

28. (canceled)

29. The method of claim 25, wherein the amplifying takes place in the presence or the absence of the endonuclease.

30. The method of claim 25, wherein amplifying the cf-ctNA comprises isothermal amplification or thermal cycling.

31. (canceled)

32. The method of claim 25, wherein the detecting comprises analyzing the amplified nucleic acid with an assay capable of distinguishing cf-ctNA from non-target or wildtype NA.

33. (canceled)

34. The method of claim 25, wherein the endonuclease is an Argonaute enzyme, a Thermus thermophiles Argonaute enzyme (TtAgo), or a Pyrococcus furiosus Argonaute enzyme (PfAgo).

35. (canceled)

36. (canceled)

37. The method of claim 25, wherein the sample is a liquid biopsy.

38. The method of claim 25, wherein the cf-ctNA comprises a mutation or is a variant associated with a disease.

39. The method of claim 25, wherein the guide nucleic acid is incubated with the endonuclease prior to incubation with the cf-ctNA and non-target or wildtype NA.

40. The method of claim 25, wherein the cf-ctNA does not comprise a protospacer adjacent motif.

41. (canceled)

42. The method of claim 25, wherein the cf-ctNA is a DNA or a RNA.

43. (canceled)

44. The method of claim 25, wherein the non-target or wildtype NA is non-cell free circulating tumor nucleic acid (non-cf-ctNA).

45. The method of claim 25, wherein at least the contacting and amplifying steps are performed in the presence of a buffer comprising at least one reagent selected from the group consisting of: (a) about 0.2 M to about 2 M betaine; (b) about 0.1 mM to about 2.5 mM dNTP, and about 2 to about 16 mM Mg²⁺.

46. (canceled)

47. (canceled)

48. A method of detecting a molecule in a sample, comprising:
a. contacting the sample with a first antibody having an affinity for a first epitope on the molecule, wherein in the presence of the molecule a molecule-first antibody complex is formed;
b. contacting the sample with a probe comprising (i) a second antibody having an affinity for a second epitope on the molecule, (ii) a guide nucleic acid, and (iii) optionally a linker linking the second antibody to the guide nucleic acid, wherein in the presence of the target molecule-first antibody complex form a complex of the probe and the target molecule-first antibody complex is formed;
c. contacting the sample with a target nucleic acid comprising a first portion labeled with a dye, a second portion labeled with a quencher, and a sequence at least partially complementary to a sequence of the guide nucleic acid, wherein in the presence of the detectable complex, the guide nucleic acid of the probe hybridizes to the target nucleic acid to form a guide-target complex;
d. contacting the sample with an endonuclease having an affinity for the guide-target complex; and
e. detecting a signal related to the dye.

49. The method of claim 48, further comprising quantitating the detected signal.

50. The method of claim 48, wherein the first antibody is tethered to a substrate and wherein the tethered first antibody is optionally immobile, and the substrate optionally comprises a microfluidics device, a microchip slide, a resin, or a polymer.

51. (canceled)

52. (canceled)

53. The method of claim 48, further comprising removing molecules not bound by the first antibody.

54. (canceled)

55. The method of claim 48, wherein the second antibody and the guide nucleic acid are directly conjugated or joined by a linker.

56. (canceled)

57. The method of claim 48, wherein the guide nucleic acid comprises at least one mismatch in relation to the target nucleic acid sequence.

58. A method of enriching a target nucleic acid sequence for next-generation sequencing comprising:
a. protecting, in a population of nucleic acids, a first end of the target nucleic acid with at least a first inactive Argonaute-guide complex and a second end of the target nucleic acid with at least a second inactive Argonaute-guide complex;
b. digesting the unprotected nucleic acid with an exonuclease; and
c. detecting the protected nucleic acid.

59. The method of claim 58, wherein the target nucleic acid is double stranded.

60. The method of claim 59, wherein the at least a first inactive Argonaute-guide complex comprises at least two inactive Argonaute proteins and the at least a second inactive Argonaute-guide complex comprises at least two inactive Argonaute proteins.

61. (canceled)

62. The method of claim 58, wherein the first inactive Argonaute-guide complex comprises inactive Argonaute protein complexed with a first pair of DNA guides, and the second inactive Argonaute-guide complex comprises inactive Argonaute protein complexed with a second pair of DNA guides.

63. (canceled)

64. (canceled)

65. (canceled)

66. The method of claim 58, wherein the target nucleic acid is from a pathogen.

67. The method of claim 58, wherein the population of nucleic acids is isolated from an organism, a soil, a water, or a food or a combination a thereof, and the target nucleic acid comprises a sequence from a mitochondrial genome of the organism or a sequence foreign to the organism, a sequence foreign to the organism, or one or more microbial nucleic acid sequences, the method further comprising characterizing a microbiome of the organism.

68. (canceled)

69. (canceled)

70. (canceled)

71. The method of claim 58, wherein the detecting comprises hybridization, spectrophotometry, sequencing, electrophoresis, amplification, fluorescence, chromatography, or a combination thereof.

72. A method of suppressing amplification of non-target nucleic acid by including in a reaction mixture an inactive Argonaute protein-guide complex, wherein the guide is sufficiently complementary to the non-target nucleic acid to form a non-target nucleic acid-inactivated Argonaute protein complex.


SEQUENCE LISTING (178 sequences)

SEQ ID NO: 1 - DNA, 47 bp, Homo sapiens
ataaacttgtggtagttggagctggtggcgtaggcaagagtgccttg

SEQ ID NO: 2 - DNA, 47 bp, Homo sapiens
ataaacttgtggtagttggagctgatggcgtaggcaagagtgccttg

SEQ ID NO: 3 - DNA, 19 bp, Artificial Sequence
  Description of Artificial Sequence Synthetic oligonucleotide
tccaccagcgccaactacc

SEQ ID NO: 4 - DNA, 29 bp, Homo sapiens
ggtagttggagctggtggcgtaggcaaga

SEQ ID NO: 5 - DNA, 29 bp, Homo sapiens
tcttgcctacgccaccagctccaactacc

SEQ ID NO: 6 - DNA, 19 bp, Artificial Sequence
  Description of Artificial Sequence Synthetic oligonucleotide
tctggtggcataggcaaga

SEQ ID NO: 7 - DNA, 19 bp, Artificial Sequence
  Description of Artificial Sequence Synthetic oligonucleotide
tccaccagcgccaactacc

SEQ ID NO: 8 - DNA, 29 bp, Homo sapiens
ggtagttggagctgatggcgtaggcaaga

SEQ ID NO: 9 - DNA, 29 bp, Homo sapiens
tcttgcctacgccatcagctccaactacc

SEQ ID NO: 10 - DNA, 19 bp, Artificial Sequence
  Description of Artificial Sequence Synthetic oligonucleotide
tctggtggcataggcaaga

SEQ ID NO: 11 - DNA, 12 bp, Homo sapiens
gagctggtggcg

SEQ ID NO: 12 - DNA, 12 bp, Homo sapiens
gagctgrtggcg

SEQ ID NO: 13 - DNA, 100 bp, Homo sapiens
aacgtactggtgaaaacaccgcagcatgtcaagatcacagattttgggctggccaaactgctgggtgcggaagagaaagaataccatgcagaaggaggca

SEQ ID NO: 14 - DNA, 100 bp, Homo sapiens
aacgtactggtgaaaacaccgcagcatgtcaagatcacagattttgggcgggccaaactgctgggtgcggaagagaaagaataccatgcagaaggaggca

SEQ ID NO: 15 - DNA, 18 bp, Artificial Sequence
  Description of Artificial Sequence Synthetic oligonucleotide
attggccagaccaaaatc

SEQ ID NO: 16 - DNA, 18 bp, Artificial Sequence
  Description of Artificial Sequence Synthetic oligonucleotide
atggccagcacaaaatct

SEQ ID NO: 17 - DNA, 18 bp, Artificial Sequence
  Description of Artificial Sequence Synthetic oligonucleotide
aggccagccaaaaatctg

SEQ ID NO: 18 - DNA, 18 bp, Artificial Sequence
  Description of Artificial Sequence Synthetic oligonucleotide
agccagcccgaaatctgt

SEQ ID NO: 19 - DNA, 18 bp, Artificial Sequence
  Description of Artificial Sequence Synthetic oligonucleotide
accagcccagaatctgtg

SEQ ID NO: 20 - DNA, 18 bp, Artificial Sequence
  Description of Artificial Sequence Synthetic oligonucleotide
acagcccaagatctgtga

SEQ ID NO: 21 - DNA, 18 bp, Artificial Sequence
  Description of Artificial Sequence Synthetic oligonucleotide
aagcccaaagtctgtgat

SEQ ID NO: 22 - DNA, 20 bp, Artificial Sequence
  Description of Artificial Sequence Synthetic oligonucleotide
cctggctccccagaaccacg

SEQ ID NO: 23 - RNA, 45 nt, Zika virus
caaaccgucgucguucuggggagccaggaaggagccguucacacg

SEQ ID NO: 24 - DNA, 20 bp, Artificial Sequence
  Description of Artificial Sequence Synthetic oligonucleotide
cctggctccccagaaccacg

SEQ ID NO: 25 - RNA, 45 nt, Zika virus
caaacugucgugguucuagggagucaagaaggagcaguucacacg

SEQ ID NO: 26 - DNA, 100 bp, Homo sapiens
aaatgattctgaattagctgtatcgtcaaggcactcttgcctacgccaccagctccaactaccacaagtttatattcagtcattttcagcaggccttata

SEQ ID NO: 27 - DNA, 15 bp, Artificial Sequence
  Description of Artificial Sequence Synthetic oligonucleotide
ttagttggagctggt

SEQ ID NO: 28 - DNA, 15 bp, Artificial Sequence
  Description of Artificial Sequence Synthetic oligonucleotide
tagttggagctggtg

SEQ ID NO: 29 - DNA, 15 bp, Artificial Sequence
  Description of Artificial Sequence Synthetic oligonucleotide
tgttggagctggtgg

SEQ ID NO: 30 - DNA, 15 bp, Artificial Sequence
  Description of Artificial Sequence Synthetic oligonucleotide
tttggagctggtggc

SEQ ID NO: 31 - DNA, 15 bp, Artificial Sequence
  Description of Artificial Sequence Synthetic oligonucleotide
ttggagctggtggcg

SEQ ID NO: 32 - DNA, 15 bp, Artificial Sequence
  Description of Artificial Sequence Synthetic oligonucleotide
tggagctggtggcgt

SEQ ID NO: 33 - DNA, 15 bp, Artificial Sequence
  Description of Artificial Sequence Synthetic oligonucleotide
tgagctggtggcgta

SEQ ID NO: 34 - DNA, 15 bp, Artificial Sequence
  Description of Artificial Sequence Synthetic oligonucleotide
tagctggtggcgtag

SEQ ID NO: 35 - DNA, 15 bp, Artificial Sequence
  Description of Artificial Sequence Synthetic oligonucleotide
tgctggtggcgtagg

SEQ ID NO: 36 - DNA, 15 bp, Artificial Sequence
  Description of Artificial Sequence Synthetic oligonucleotide
tctggtggcgtaggc

SEQ ID NO: 37 - DNA, 15 bp, Artificial Sequence
  Description of Artificial Sequence Synthetic oligonucleotide
ttggtggcgtaggca

SEQ ID NO: 38 - DNA, 100 bp, Homo sapiens
aaatgattctgaattagctgtatcgtcaaggcactcttgcctacgccatcagctccaactaccacaagtttatattcagtcattttcagcaggccttata

SEQ ID NO: 39 - DNA, 16 bp, Artificial Sequence
  Description of Artificial Sequence Synthetic oligonucleotide
tcagcagtttggccag

SEQ ID NO: 40 - DNA, 16 bp, Artificial Sequence
  Description of Artificial Sequence Synthetic oligonucleotide
tagcagtttggccagc

SEQ ID NO: 41 - DNA, 16 bp, Artificial Sequence
  Description of Artificial Sequence Synthetic oligonucleotide
tgcagtttggccagcc

SEQ ID NO: 42 - DNA, 16 bp, Artificial Sequence
  Description of Artificial Sequence Synthetic oligonucleotide
tcagtttggccagccc

SEQ ID NO: 43 - DNA, 16 bp, Artificial Sequence
  Description of Artificial Sequence Synthetic oligonucleotide
tagtttggccagccca

SEQ ID NO: 44 - DNA, 16 bp, Artificial Sequence
  Description of Artificial Sequence Synthetic oligonucleotide
tgtttggccagcccaa

SEQ ID NO: 45 - DNA, 16 bp, Artificial Sequence
  Description of Artificial Sequence Synthetic oligonucleotide
ttttggccagcccaaa

SEQ ID NO: 46 - DNA, 16 bp, Artificial Sequence
  Description of Artificial Sequence Synthetic oligonucleotide
tttggccagcccaaaa

SEQ ID NO: 47 - DNA, 16 bp, Artificial Sequence
  Description of Artificial Sequence Synthetic oligonucleotide
ttggccagcccaaaat

SEQ ID NO: 48 - DNA, 16 bp, Artificial Sequence
  Description of Artificial Sequence Synthetic oligonucleotide
tggccagcccaaaatc

SEQ ID NO: 49 - DNA, 16 bp, Artificial Sequence
  Description of Artificial Sequence Synthetic oligonucleotide
tgccagcccaaaatct

SEQ ID NO: 50 - DNA, 16 bp, Artificial Sequence
  Description of Artificial Sequence Synthetic oligonucleotide
tccagcccaaaatctg

SEQ ID NO: 51 - DNA, 16 bp, Artificial Sequence
  Description of Artificial Sequence Synthetic oligonucleotide
tcagcccaaaatctgt

SEQ ID NO: 52 - DNA, 16 bp, Artificial Sequence
  Description of Artificial Sequence Synthetic oligonucleotide
tagcccaaaatctgtg

SEQ ID NO: 53 - DNA, 98 bp, Homo sapiens
gcctccttctgcatggtattctttctcttccgcacccagcagtttggccagcccaaaatctgtgatcttgacatgctgcggtgttttcaccagtacgt

SEQ ID NO: 54 - DNA, 16 bp, Artificial Sequence
  Description of Artificial Sequence Synthetic oligonucleotide
ttggccaaactgctgg

SEQ ID NO: 55 - DNA, 16 bp, Artificial Sequence
  Description of Artificial Sequence Synthetic oligonucleotide
tctggccaaactgctg

SEQ ID NO: 56 - DNA, 16 bp, Artificial Sequence
  Description of Artificial Sequence Synthetic oligonucleotide
tgctggccaaactgct

SEQ ID NO: 57 - DNA, 16 bp, Artificial Sequence
  Description of Artificial Sequence Synthetic oligonucleotide
tggctggccaaactgc

SEQ ID NO: 58 - DNA, 16 bp, Artificial Sequence
  Description of Artificial Sequence Synthetic oligonucleotide
tgggctggccaaactg

SEQ ID NO: 59 - DNA, 16 bp, Artificial Sequence
  Description of Artificial Sequence Synthetic oligonucleotide
ttgggctggccaaact

SEQ ID NO: 60 - DNA, 16 bp, Artificial Sequence
  Description of Artificial Sequence Synthetic oligonucleotide
tttgggctggccaaac

SEQ ID NO: 61 - DNA, 16 bp, Artificial Sequence
  Description of Artificial Sequence Synthetic oligonucleotide
ttttgggctggccaaa

SEQ ID NO: 62 - DNA, 16 bp, Artificial Sequence
  Description of Artificial Sequence Synthetic oligonucleotide
tttttgggctggccaa

SEQ ID NO: 63 - DNA, 16 bp, Artificial Sequence
  Description of Artificial Sequence Synthetic oligonucleotide
tattttgggctggcca

SEQ ID NO: 64 - DNA, 16 bp, Artificial Sequence
  Description of Artificial Sequence Synthetic oligonucleotide
tgattttgggctggcc

SEQ ID NO: 65 - DNA, 16 bp, Artificial Sequence
  Description of Artificial Sequence Synthetic oligonucleotide
tagattttgggctggc

SEQ ID NO: 66 - DNA, 16 bp, Artificial Sequence
  Description of Artificial Sequence Synthetic oligonucleotide
tcagattttgggctgg

SEQ ID NO: 67 - DNA, 16 bp, Artificial Sequence
  Description of Artificial Sequence Synthetic oligonucleotide
tacagattttgggctg

SEQ ID NO: 68 - DNA, 98 bp, Homo sapiens
gcctccttctgcatggtattctttctcttccgcacccagcagtttggcccgcccaaaatctgtgatcttgacatgctgcggtgttttcaccagtacgt

SEQ ID NO: 69 - DNA, 16 bp, Artificial Sequence
  Description of Artificial Sequence Synthetic oligonucleotide
tgcgtgatgagctgca

SEQ ID NO: 70 - DNA, 16 bp, Artificial Sequence
  Description of Artificial Sequence Synthetic oligonucleotide
ttgcgtgatgagctgc

SEQ ID NO: 71 - DNA, 16 bp, Artificial Sequence
  Description of Artificial Sequence Synthetic oligonucleotide
tctgcgtgatgagctg

SEQ ID NO: 72 - DNA, 16 bp, Artificial Sequence
  Description of Artificial Sequence Synthetic oligonucleotide
tgctgcgtgatgagct

SEQ ID NO: 73 - DNA, 16 bp, Artificial Sequence
  Description of Artificial Sequence Synthetic oligonucleotide
tagctgcgtgatgagc

SEQ ID NO: 74 - DNA, 16 bp, Artificial Sequence
  Description of Artificial Sequence Synthetic oligonucleotide
tgagctgcgtgatgag

SEQ ID NO: 75 - DNA, 16 bp, Artificial Sequence
  Description of Artificial Sequence Synthetic oligonucleotide
ttgagctgcgtgatga

SEQ ID NO: 76 - DNA, 16 bp, Artificial Sequence
  Description of Artificial Sequence Synthetic oligonucleotide
tatgagctgcgtgatg

SEQ ID NO: 77 - DNA, 16 bp, Artificial Sequence
  Description of Artificial Sequence Synthetic oligonucleotide
tcatgagctgcgtgat

SEQ ID NO: 78 - DNA, 16 bp, Artificial Sequence
  Description of Artificial Sequence Synthetic oligonucleotide
tgcatgagctgcgtga

SEQ ID NO: 79 - DNA, 16 bp, Artificial Sequence
  Description of Artificial Sequence Synthetic oligonucleotide
tggcatgagctgcgtg

SEQ ID NO: 80 - DNA, 100 bp, Homo sapiens
ctttgtgttcccggacatagtccaggaggcagccgaagggcatgagctgcgtgatgagctgcacggtggaggtgaggcagatgcccagcaggcggcacac

SEQ ID NO: 81 - DNA, 16 bp, Artificial Sequence
  Description of Artificial Sequence Synthetic oligonucleotide
ttgcagctcatcacgc

SEQ ID NO: 82 - DNA, 16 bp, Artificial Sequence
  Description of Artificial Sequence Synthetic oligonucleotide
tgcagctcatcacgca

SEQ ID NO: 83 - DNA, 16 bp, Artificial Sequence
  Description of Artificial Sequence Synthetic oligonucleotide
tcagctcatcacgcag

SEQ ID NO: 84 - DNA, 16 bp, Artificial Sequence
  Description of Artificial Sequence Synthetic oligonucleotide
tagctcatcacgcagc

SEQ ID NO: 85 - DNA, 16 bp, Artificial Sequence
  Description of Artificial Sequence Synthetic oligonucleotide
tgctcatcacgcagct

SEQ ID NO: 86 - DNA, 16 bp, Artificial Sequence
  Description of Artificial Sequence Synthetic oligonucleotide
tctcatcacgcagctc

SEQ ID NO: 87 - DNA, 16 bp, Artificial Sequence
  Description of Artificial Sequence Synthetic oligonucleotide
ttcatcacgcagctca

SEQ ID NO: 88 - DNA, 16 bp, Artificial Sequence
  Description of Artificial Sequence Synthetic oligonucleotide
tcatcacgcagctcat

SEQ ID NO: 89 - DNA, 16 bp, Artificial Sequence
  Description of Artificial Sequence Synthetic oligonucleotide
tatcacgcagctcatg

SEQ ID NO: 90 - DNA, 16 bp, Artificial Sequence
  Description of Artificial Sequence Synthetic oligonucleotide
ttcacgcagctcatgc

SEQ ID NO: 91 - DNA, 16 bp, Artificial Sequence
  Description of Artificial Sequence Synthetic oligonucleotide
tcacgcagctcatgcc

SEQ ID NO: 92 - DNA, 100 bp, Homo sapiens
ctttgtgttcccggacatagtccaggaggcagccgaagggcatgagctgcatgatgagctgcacggtggaggtgaggcagatgcccagcaggcggcacac

SEQ ID NO: 93 - DNA, 16 bp, Artificial Sequence
  Description of Artificial Sequence Synthetic oligonucleotide
ttcactgtagctagac

SEQ ID NO: 94 - DNA, 16 bp, Artificial Sequence
  Description of Artificial Sequence Synthetic oligonucleotide
tttcactgtagctaga

SEQ ID NO: 95 - DNA, 16 bp, Artificial Sequence
  Description of Artificial Sequence Synthetic oligonucleotide
ttttcactgtagctag

SEQ ID NO: 96 - DNA, 16 bp, Artificial Sequence
  Description of Artificial Sequence Synthetic oligonucleotide
tatttcactgtagcta

SEQ ID NO: 97 - DNA, 16 bp, Artificial Sequence
  Description of Artificial Sequence Synthetic oligonucleotide
tgatttcactgtagct

SEQ ID NO: 98 - DNA, 16 bp, Artificial Sequence
  Description of Artificial Sequence Synthetic oligonucleotide
tagatttcactgtagc

SEQ ID NO: 99 - DNA, 16 bp, Artificial Sequence
  Description of Artificial Sequence Synthetic oligonucleotide
tgagatttcactgtag

SEQ ID NO: 100 - DNA, 16 bp, Artificial Sequence
  Description of Artificial Sequence Synthetic oligonucleotide
tcgagatttcactgta

SEQ ID NO: 101 - DNA, 16 bp, Artificial Sequence
  Description of Artificial Sequence Synthetic oligonucleotide
ttcgagatttcactgt

SEQ ID NO: 102 - DNA, 16 bp, Artificial Sequence
  Description of Artificial Sequence Synthetic oligonucleotide
tatcgagatttcactg

SEQ ID NO: 103 - DNA, 16 bp, Artificial Sequence
  Description of Artificial Sequence Synthetic oligonucleotide
tcatcgagatttcact

SEQ ID NO: 104 - DNA, 100 bp, Homo sapiens
tggatccagacaactgttcaaactgatgggacccactccatcgagatttcactgtagctagaccaaaatcacctatttttactgtgaggtcttcatgaag

SEQ ID NO: 105 - DNA, 16 bp, Artificial Sequence
  Description of Artificial Sequence Synthetic oligonucleotide
tgtctagctacagtga

SEQ ID NO: 106 - DNA, 16 bp, Artificial Sequence
  Description of Artificial Sequence Synthetic oligonucleotide
ttctagctacagtgaa

SEQ ID NO: 107 - DNA, 16 bp, Artificial Sequence
  Description of Artificial Sequence Synthetic oligonucleotide
tctagctacagtgaaa

SEQ ID NO: 108 - DNA, 16 bp, Artificial Sequence
  Description of Artificial Sequence Synthetic oligonucleotide
ttagctacagtgaaat

SEQ ID NO: 109 - DNA, 16 bp, Artificial Sequence
  Description of Artificial Sequence Synthetic oligonucleotide
tagctacagtgaaatc

SEQ ID NO: 110 - DNA, 16 bp, Artificial Sequence
  Description of Artificial Sequence Synthetic oligonucleotide
tgctacagtgaaatct

SEQ ID NO: 111 - DNA, 16 bp, Artificial Sequence
  Description of Artificial Sequence Synthetic oligonucleotide
tctacagtgaaatctc

SEQ ID NO: 112 - DNA, 16 bp, Artificial Sequence
  Description of Artificial Sequence Synthetic oligonucleotide
ttacagtgaaatctcg

SEQ ID NO: 113 - DNA, 16 bp, Artificial Sequence
  Description of Artificial Sequence Synthetic oligonucleotide
tacagtgaaatctcga

SEQ ID NO: 114 - DNA, 16 bp, Artificial Sequence
  Description of Artificial Sequence Synthetic oligonucleotide
tcagtgaaatctcgat

SEQ ID NO: 115 - DNA, 16 bp, Artificial Sequence
  Description of Artificial Sequence Synthetic oligonucleotide
tagtgaaatctcgatg

SEQ ID NO: 116 - DNA, 100 bp, Homo sapiens
tggatccagacaactgttcaaactgatgggacccactccatcgagatttctctgtagctagaccaaaatcacctatttttactgtgaggtcttcatgaag

SEQ ID NO: 117 - DNA, 19 bp, Artificial Sequence
  Description of Artificial Sequence Synthetic oligonucleotide
tccagctccaactaccaca

SEQ ID NO: 118 - DNA, 19 bp, Artificial Sequence
  Description of Artificial Sequence Synthetic oligonucleotide
taccagctccaactaccac

SEQ ID NO: 119 - DNA, 19 bp, Artificial Sequence
  Description of Artificial Sequence Synthetic oligonucleotide
tcaccagctccaactacca

SEQ ID NO: 120 - DNA, 19 bp, Artificial Sequence
  Description of Artificial Sequence Synthetic oligonucleotide
tccaccagctccaactacc

SEQ ID NO: 121 - DNA, 19 bp, Artificial Sequence
  Description of Artificial Sequence Synthetic oligonucleotide
tgccaccagctccaactac

SEQ ID NO: 122 - DNA, 19 bp, Artificial Sequence
  Description of Artificial Sequence Synthetic oligonucleotide
tcgccaccagctccaacta

SEQ ID NO: 123 - DNA, 19 bp, Artificial Sequence
  Description of Artificial Sequence Synthetic oligonucleotide
tacgccaccagctccaact

SEQ ID NO: 124 - DNA, 19 bp, Artificial Sequence
  Description of Artificial Sequence Synthetic oligonucleotide
ttacgccaccagctccaac

SEQ ID NO: 125 - DNA, 19 bp, Artificial Sequence
  Description of Artificial Sequence Synthetic oligonucleotide
tctacgccaccagctccaa

SEQ ID NO: 126 - DNA, 19 bp, Artificial Sequence
  Description of Artificial Sequence Synthetic oligonucleotide
tcctacgccaccagctcca

SEQ ID NO: 127 - DNA, 19 bp, Artificial Sequence
  Description of Artificial Sequence Synthetic oligonucleotide
tgcctacgccaccagctcc

SEQ ID NO: 128 - DNA, 19 bp, Artificial Sequence
  Description of Artificial Sequence Synthetic oligonucleotide
ttgcctacgccaccagctc

SEQ ID NO: 129 - DNA, 19 bp, Artificial Sequence
  Description of Artificial Sequence Synthetic oligonucleotide
tttgcctacgccaccagct

SEQ ID NO: 130 - DNA, 19 bp, Artificial Sequence
  Description of Artificial Sequence Synthetic oligonucleotide
tcttgcctacgccaccagc

SEQ ID NO: 131 - DNA, 19 bp, Artificial Sequence
  Description of Artificial Sequence Synthetic oligonucleotide
ttcttgcctacgccaccag

SEQ ID NO: 132 - DNA, 100 bp, Homo sapiens; identical to SEQ ID NO: 26

SEQ ID NO: 133 - DNA, 18 bp, Artificial Sequence
  Description of Artificial Sequence Synthetic oligonucleotide
ttagttggagctggtggc

SEQ ID NO: 134 - DNA, 18 bp, Artificial Sequence
  Description of Artificial Sequence Synthetic oligonucleotide
tagttggagctggtggcg

SEQ ID NO: 135 - DNA, 18 bp, Artificial Sequence
  Description of Artificial Sequence Synthetic oligonucleotide
tgttggagctggtggcgt

SEQ ID NO: 136 - DNA, 18 bp, Artificial Sequence
  Description of Artificial Sequence Synthetic oligonucleotide
tttggagctggtggcgta

SEQ ID NO: 137 - DNA, 18 bp, Artificial Sequence
  Description of Artificial Sequence Synthetic oligonucleotide
ttggagctggtggcgtag

SEQ ID NO: 138 - DNA, 18 bp, Artificial Sequence
  Description of Artificial Sequence Synthetic oligonucleotide
tggagctggtggcgtagg

SEQ ID NO: 139 - DNA, 18 bp, Artificial Sequence
  Description of Artificial Sequence Synthetic oligonucleotide
tgagctggtggcgtaggc

SEQ ID NO: 140 - DNA, 18 bp, Artificial Sequence
  Description of Artificial Sequence Synthetic oligonucleotide
tagctggtggcgtaggca

SEQ ID NO: 141 - DNA, 18 bp, Artificial Sequence
  Description of Artificial Sequence Synthetic oligonucleotide
tgctggtggcgtaggcaa

SEQ ID NO: 142 - DNA, 18 bp, Artificial Sequence
  Description of Artificial Sequence Synthetic oligonucleotide
tctggtggcgtaggcaag

SEQ ID NO: 143 - DNA, 18 bp, Artificial Sequence
  Description of Artificial Sequence Synthetic oligonucleotide
ttggtggcgtaggcaaga

SEQ ID NO: 144 - DNA, 100 bp, Homo sapiens; identical to SEQ ID NO: 38

SEQ ID NO: 145 - DNA, 16 bp, Artificial Sequence
  Description of Artificial Sequence Synthetic oligonucleotide
ttctcttaattccttg

SEQ ID NO: 146 - DNA, 80 bp, Homo sapiens
ggtgagaaagttaaaattcccgtcgctatcaaggaattaagagaagcaacatctccgaaagccaacaaggaaatcctcga

SEQ ID NO: 147 - DNA, 65 bp, Homo sapiens
ggtgagaaagttaaaattcccgtcgctatcaaaacatctccgaaagccaacaaggaaatcctcga

SEQ ID NO: 148 - DNA, 65 bp, Homo sapiens
ggtgagaaagttaaaattcccgtcgctatcaagacatctccgaaagccaacaaggaaatcctcga

SEQ ID NO: 149 - DNA, 62 bp, Homo sapiens
ggtgagaaagttaaaattcccgtcgctatcaaggaatcgaaagccaacaaggaaatcctcga

SEQ ID NO: 150 - DNA, 65 bp, Homo sapiens
ggtgagaaagttaaaattcccgtcgctatcaaggaatctccgaaagccaacaaggaaatcctcga

SEQ ID NO: 151 - DNA, 71 bp, Homo sapiens
ggtgagaaagttaaaattcccgtcgctatcaaggaaccaacatctccgaaagccaacaaggaaatcctcga

SEQ ID NO: 152 - DNA, 62 bp, Homo sapiens
ggtgagaaagttaaaattcccgtcgctatcaaggttccgaaagccaacaaggaaatcctcga

SEQ ID NO: 153 - DNA, 62 bp, Homo sapiens; identical to SEQ ID NO: 152

SEQ ID NO: 154 - DNA, 62 bp, Homo sapiens
ggtgagaaagttaaaattcccgtcgctatcaaggaaccgaaagccaacaaggaaatcctcga

SEQ ID NO: 155 - DNA, 16 bp, Artificial Sequence
  Description of Artificial Sequence Synthetic oligonucleotide
taagagaagcaacatc

SEQ ID NO: 156 - DNA, 80 bp, Homo sapiens
tcgaggatttccttgttggctttcggagatgttgcttctcttaattccttgatagcgacgggaattttaactttctcacc

SEQ ID NO: 157 - DNA, 65 bp, Homo sapiens
tcgaggatttccttgttggctttcggagatgttttgatagcgacgggaattttaactttctcacc

SEQ ID NO: 158 - DNA, 65 bp, Homo sapiens
tcgaggatttccttgttggctttcggagatgtcttgatagcgacgggaattttaactttctcacc

SEQ ID NO: 159 - DNA, 62 bp, Homo sapiens
tcgaggatttccttgttggctttcgattccttgatagcgacgggaattttaactttctcacc

SEQ ID NO: 160 - DNA, 65 bp, Homo sapiens
tcgaggatttccttgttggctttcggagattccttgatagcgacgggaattttaactttctcacc

SEQ ID NO: 161 - DNA, 71 bp, Homo sapiens
tcgaggatttccttgttggctttcggagatgttggttccttgatagcgacgggaattttaactttctcacc

SEQ ID NO: 162 - DNA, 62 bp, Homo sapiens
tcgaggatttccttgttggctttcggaaccttgatagcgacgggaattttaactttctcacc

SEQ ID NO: 163 - DNA, 62 bp, Homo sapiens; identical to SEQ ID NO: 162

SEQ ID NO: 164 - DNA, 62 bp, Homo sapiens
tcgaggatttccttgttggctttcggttccttgatagcgacgggaattttaactttctcacc

SEQ ID NO: 165 - DNA, 25 bp, Artificial Sequence
  Description of Artificial Sequence Synthetic primer
aggcctgctgaaaatgactgaatat

SEQ ID NO: 166 - DNA, 22 bp, Artificial Sequence
  Description of Artificial Sequence Synthetic primer
gctgtatcgtcaaggcactctt

SEQ ID NO: 167 - DNA, 16 bp, Artificial Sequence
  Description of Artificial Sequence Synthetic probe
ttggagctggtggcgt

SEQ ID NO: 168 - DNA, 15 bp, Artificial Sequence
  Description of Artificial Sequence Synthetic probe
tggagctgatggcgt

SEQ ID NO: 169 - DNA, 16 bp, Artificial Sequence
  Description of Artificial Sequence Synthetic probe
ttggagctcgtggcgt

SEQ ID NO: 170 - DNA, 13 bp, Artificial Sequence
  Description of Artificial Sequence Synthetic probe
acgccaacagctc

SEQ ID NO: 171 - DNA, 21 bp, Artificial Sequence
  Description of Artificial Sequence Synthetic primer
aaggtactggtggagtatttg

SEQ ID NO: 172 - DNA, 22 bp, Artificial Sequence
  Description of Artificial Sequence Synthetic primer
gtactcatgaaaatggtcagag

SEQ ID NO: 173 - DNA, 17 bp, Artificial Sequence
  Description of Artificial Sequence Synthetic primer
tattataaggcctgctg

SEQ ID NO: 174 - DNA, 17 bp, Artificial Sequence
  Description of Artificial Sequence Synthetic primer
ttggatcatattcgtcc

SEQ ID NO: 175 - DNA, 42 bp, Artificial Sequence
  Description of Artificial Sequence Synthetic primer
accttctaccctcagaaggtataaacttgtggtagttggagc

SEQ ID NO: 176 - DNA, 15 bp, Artificial Sequence
  Description of Artificial Sequence Synthetic primer
gcaagagtgccttga

SEQ ID NO: 177 - DNA, 31 bp, Artificial Sequence
  Description of Artificial Sequence Synthetic primer
tggcgtaggcatgattctgaattagctgtat

SEQ ID NO: 178 - DNA, 17 bp, Artificial Sequence
  Description of Artificial Sequence Synthetic oligonucleotide
cctacgccaccagctcc